(19) United States
(12) Patent Application Publication
(10) Pub. No.: US 2005/0034317 A1
(43) Pub. Date: Feb. 17, 2005

(54) Wearable-article sizing system

(76) Inventors: Ted Burandt, Phoenix, AZ (US); Charlotte Burandt, Phoenix, AZ (US); Jason Burandt, Phoenix, AZ (US)

Correspondence Address: STONEMAN LAW OFFICES, LTD, 3113 NORTH 3RD STREET, PHOENIX, AZ, 85012, US

(21) Appl. No.: 10/918,571

(22) Filed: Aug. 12, 2004

Related U.S. Application Data
(60) Provisional application No. 60/495,338, filed on Aug. 14, 2003.

(51) Int. Cl. A61B 5/103

(52) U.S. Cl. 33/512

(57) ABSTRACT

A wearable-article sizing system which allows customers to measure their wrist, ankle, finger, toe, head, neck, or watch-lug size by electronically downloading and printing an apparatus with instructions for measuring their size, and allows Internet vendors or other electronic vendors to provide the value-added beneficial service of custom sizing wristwatches, necklaces, rings, bracelets, or hats before they are shipped to the customers.

### CROSS-REFERENCE TO RELATED APPLICATION

The present application claims priority from and is related to prior provisional application Ser. No. 60/495,338, filed Aug. 14, 2003, entitled “WRISTBAND SIZING SYSTEM”, the contents of which are incorporated herein by this reference and are not admitted to be prior art with respect to the present invention by the mention in this cross-reference section.

### BACKGROUND

This invention relates to providing a system and method for providing properly sized wearable articles (such as wristwatch wristbands, rings, hats, bracelets including ankle bracelets, and necklaces) and related systems and methods. In the past, mail order, catalog, Internet, and other types of vendors that ship wearable articles to customers have shipped such articles without custom sizing the articles to fit the customer. As a result, when the customer receives a watch (for example) from the vendor, the customer must then take the additional step of getting the watchband sized properly, usually at additional cost and inconvenience to the customer. Customers may be more reluctant to order a watch from an Internet shopping site because of the added inconvenience of having to size the watch after receiving it.

Further, customers often do not know their wrist size (ring size, hat size, etc.) or how to measure it. Vendors that sell watches via a shopping website, and would otherwise have the capability of sizing watches, cannot do so because they do not have a means of obtaining the customer's proper size, thus losing potential extra income for the value-added service of custom sizing the watch (or other article) before it is shipped to the customer.

Therefore, a need exists for a system to help a customer have an Internet-purchased wearable article pre-sized and/or to help the vendor or broker charge for the sizing to add value to the article purchase.

### OBJECTS AND FEATURES OF THE INVENTION

A primary object and feature of this invention is to overcome the above-mentioned problems and meet the above-mentioned needs. Another primary object and feature of the present invention is to provide a wearable-article sizing system.

Another primary object and feature of the present invention is to provide a wristband sizing system. Another primary object and feature of the present invention is to provide a hat sizing system. Another primary object and feature of the present invention is to provide a ring sizing system. Another primary object and feature of the present invention is to provide a necklace sizing system. Another primary object and feature of the present invention is to provide a watchband lug sizing system.

Another primary object and feature of the present invention is to provide a system that allows customers to measure their wrist size. Another primary object and feature of the present invention is to provide a system that allows customers to measure their hat size. Another primary object and feature of the present invention is to provide a system that allows customers to measure their ring size. Another primary object and feature of the present invention is to provide a system that allows customers to measure their preferred necklace size. Another primary object and feature of the present invention is to provide a system that allows customers to measure their watchband lug size.

It is a further object and feature of the present invention to provide such a system that allows customers to download and print a kit comprising an apparatus and instructions for measuring their size.

It is a further object and feature of the present invention to provide such a system that allows vendors to provide the added beneficial service to customers of custom sizing wearable-articles before they are shipped to customers.

It is a further object and feature of the present invention to provide such a system that allows vendors to obtain additional revenue from the value-added service of custom sizing wearable articles before they are shipped to the customer.

A further primary object and feature of the present invention is to provide such a system that is efficient, inexpensive, and handy. Other objects and features of this invention will become apparent with reference to the following descriptions.

### SUMMARY OF THE INVENTION

In accordance with a preferred embodiment hereof, this invention provides a size measuring system, for allowing at least one customer to receive at least one properly sized wearable article shipped from at least one vendor, comprising: encircler means for encircling at least one article-wearable body part of the at least one customer; and first instruction means for instructing the at least one customer in the use of such encircler means to provide a measurement for the wearable article; wherein such encircler means comprises straightener means for straightening such encircler means, wherein such encircler means may be measured by a flat measuring tool; and wherein such encircler means and such first instruction means comprise downloadable computer-file means for providing a downloadable computer-file from which such encircler means and such instruction means may be printed onto a printable media. Moreover, it provides such a size measuring system, further comprising: printable media means for printing such encircler means and such first instruction means onto a printable media; wherein such encircler means and such first instruction means are printed onto such printable media means; and wherein such encircler means is cut out from such printable media means prior to use.

Further, it provides such a size measuring system, further comprising holder means for holding such encircler means in place on the at least one article-wearable body part of the customer; and holder instruction means for instructing the customer in the use of such holder means. Moreover, it provides such a size measuring system, wherein such encircler means comprises paper. Also, it provides such a size measuring system, wherein said at least one holder comprises at least one piece of adhesive tape. In addition, it provides such a size measuring system, wherein such first instruction means comprises indicia means for visually illustrating such first instruction means. Even further, this invention provides such a size measuring system, wherein such first instruction means comprises ruler instruction means for instructing the customer in the use of a ruler to measure a length of such encircler means. Further, it provides such a size measuring system, wherein such ruler instruction means comprises ruler indicia means for visually illustrating the use of a ruler to measure a length of such encircler means.

In accordance with another preferred embodiment hereof, this invention provides a size measuring system, for allowing at least one customer to receive at least one properly sized wearable article shipped from at least one vendor, comprising: at least one encircler adapted to encircle an article-wearable body part of the customer; and at least one first instruction adapted to instruct a customer in the use of such at least one encircler to provide a measurement for the wearable article; wherein such at least one encircler comprises at least one straightener adapted to straighten such at least one encircler, wherein such at least one encircler may be measured by a flat measuring tool; and wherein such at least one encircler and such at least one first instruction comprise at least one downloadable computer-file adapted to provide at least one downloadable computer-file from which such at least one encircler and such at least one instruction may be printed onto a printable media.

Even further, it provides such a size measuring system, further comprising: at least one printable media adapted to printing such at least one encircler and such at least one first instruction onto a printable media; wherein such at least one encircler and such at least one first instruction are printed onto such at least one printable media; and wherein such at least one encircler is cut out from such at least one printable media prior to use. Moreover, it provides such a size measuring system, further comprising: at least one holder adapted to hold such at least one encircler in place on the article-wearable body part of the customer; and at least one holder instruction adapted to instruct the customer in the use of such at least one holder. Additionally, it provides such a size measuring system, wherein such at least one holder comprises at least one paper clip. Also, it provides such a size measuring system, wherein such at least one encircler comprises at least one paper.

In addition, it provides such a size measuring system, wherein such at least one first instruction comprises at least one indicia adapted to visually illustrate such at least one first instruction. And, it provides such a size measuring system, wherein such at least one instructions comprises at least one ruler instructions adapted to instruct the customer in the use of a ruler to measure a length of such at least one encircler; and at least one ruler indicia adapted to visually illustrate such at least one ruler instructions.

Further, it provides such a size measuring system, wherein such at least one encircler is adapted to encircle at least one wrist of the customer. Even further, it provides such a size measuring system, wherein such at least one encircler is adapted to encircle the head of the customer. Moreover, it provides such a size measuring system, wherein such at least one encircler is adapted to encircle at least one digit of the customer. Additionally, it provides such a size measuring system, wherein such at least one encircler is adapted to encircle the neck of the customer.

In accordance with another preferred embodiment hereof, this invention provides a size measuring system, for allowing a customer to receive a properly sized wrist-wearable article shipped from a vendor, comprising: at least one encircler to encircle a wrist of the customer; wherein such at least one encircler is adapted to be straightened, wherein such at least one encircler may be measured by a linear measuring tool; and wherein such at least one encircler may be measured by a linear measuring tool. In accordance with another preferred embodiment hereof, this invention provides also, it provides such a size measuring system, further comprising at least one first instructions adapted to instruct the customer in the use of such at least one encircler. In addition, it provides such a size measuring system, further comprising at least one indicium adapted to visually assist the customer to use such at least one encircler.

In accordance with another preferred embodiment hereof, this invention provides a size measuring system, for allowing a customer to receive a properly sized head-wearable article shipped from a vendor, comprising: at least one encircler to encircle the head of the customer; wherein such at least one encircler is adapted to be straightened, wherein such at least one encircler may be measured by a linear measuring tool; and wherein such at least one encircler is structured and arranged to be downloaded electronically and printed. And, it provides such a size measuring system, further comprising at least one first instructions adapted to instruct the customer in the use of such at least one encircler. Further, it provides such a size measuring system, further comprising at least one indicium adapted to visually assist the customer to use such at least one encircler.

In accordance with another preferred embodiment hereof, this invention provides a size measuring system, for allowing a customer to receive a properly sized ring shipped from a vendor, comprising: at least one encircler to encircle a digit of the customer; wherein such at least one encircler is adapted to be straightened, wherein such at least one encircler may be measured by a linear measuring tool; and wherein such at least one encircler is structured and arranged to be downloaded electronically and printed. Even further, it provides such a size measuring system, further comprising at least one first instructions adapted to instruct the customer in the use of such at least one encircler. Moreover, it provides such a size measuring system, further comprising at least one indicium adapted to visually assist the customer to use such at least one encircler.

In accordance with another preferred embodiment hereof, this invention provides a size measuring system, for allowing a customer to receive a properly sized neck-wearable article shipped from a vendor, comprising: at least one encircler to encircle the neck of the customer; wherein such at least one encircler is adapted to be straightened, wherein such at least one encircler may be measured by a linear measuring tool; and wherein such at least one encircler is structured and arranged to be downloaded electronically and printed. Additionally, it provides such a size measuring system, further comprising at least one first instructions adapted to instruct the customer in the use of such at least one encircler. Also, it provides such a size measuring system, further comprising at least one indicium adapted to visually assist the customer to use such at least one encircler.

In accordance with another preferred embodiment hereof, this invention provides a method, for allowing a customer to receive a properly sized wearable article shipped from a vendor, comprising the steps of: electronically providing measuring instructions to the customer for a method of measuring the article-wearable body part of the customer; wherein such instructions comprise the steps of: wrapping at least one flexible object around the article-wearable body part of the customer, marking such at least one flexible object, removing such at least one flexible object from the article-wearable body part of the customer, laying such at least one flexible object out flat, measuring the marked length of such at least one flexible object; having the customer provide the measurement to the vendor; sizing at least one wearable article based on such measurement; and shipping such at least one wearable article to the customer.

In addition, it provides such a method, further comprising the step of (the customer) downloading such measuring instructions from an electronic website. And, it provides such a method, further comprising the step of (the customer) printing such measuring instruction on a printer. Further, it provides such a method, further comprising the step of (the customer) printing an encircler on a printer. Even further, it provides such a method, further comprising the step of (the customer) paying the vendor a fee to adjust the size of the wearable article.

Moreover, it provides such a method, wherein the step of sizing at least one wearable article comprises the step of sizing at least one wristband. Additionally, it provides such a method, wherein the step of sizing at least one wearable article comprises the step of sizing at least one bracelet. Also, it provides such a method, wherein the step of sizing at least one wearable article comprises the step of sizing at least one hat. In addition, it provides such a method, wherein the step of sizing at least one wearable article comprises the step of sizing at least one ring. And, it provides such a method, wherein the step of sizing at least one wearable article comprises the step of sizing at least one necklace.

In accordance with another preferred embodiment hereof, this invention provides a method, relating to providing properly sized wearable articles by vendors to customers, comprising the steps of: providing to such customer having a particular wearable-article size an electronic download apparatus for measuring such wearable-article size; and providing an upload opportunity for such customer to provide to such vendor a particular wearable-article size for such purchased wearable article. Further, it provides such a method, wherein such electronic download apparatus comprises at least one encircler. Even further, it provides such a method, wherein such electronic download apparatus comprises instructions for measuring such wearable-article size.

In accordance with another preferred embodiment hereof, this invention provides a method, for allowing at least one customer to receive at least one properly sized wearable article shipped from at least one vendor, comprising the steps of: such vendor advertising the availability of custom sizing; such vendor receiving a fee from a customer for custom sizing; such vendor providing the customer with electronically downloadable and printable instructions for measuring such customer's proper size; wherein such instructions comprise at least one encircler; such vendor receiving such customer's proper size information; such vendor adjusting such at least one wearable article to such customer's proper size; and such vendor shipping such at least one wearable article to such customer.

Moreover, it provides such a method, wherein the step of such vendor adjusting such at least one wearable article to such customer's proper size comprises the step of such vendor adjusting at least one wrist-wearable article to such customer's proper size. Additionally, it provides such a method, wherein the step of such vendor adjusting such at least one wearable article to such customer's proper size comprises the step of such vendor adjusting at least one bracelet to such customer's proper size. Also, it provides such a method, wherein the step of such vendor adjusting such at least one wearable article to such customer's proper size comprises the step of such vendor adjusting at least one head-wearable article to such customer's proper size. In addition, it provides such a method, wherein the step of such vendor adjusting such at least one wearable article to such customer's proper size comprises the step of such vendor adjusting at least one ring to such customer's proper size. And, it provides such a method, wherein the step of such vendor adjusting such at least one wearable article to such customer's proper size comprises the step of such vendor adjusting at least one neck-wearable article to such customer's proper size.

In accordance with another preferred embodiment hereof, this invention provides a size measuring system, for allowing a customer to receive a properly sized watchband shipped from a vendor, comprising: at least one scale to measure a distance between lugs of a watch; and at least one first instruction adapted to instruct a customer in the use of such at least one scale; wherein such at least one scale is structured and arranged to be downloaded electronically and printed onto a printable media. Further, it provides such a size measuring system, further comprising: at least one printable media adapted to printing such at least one scale and such at least one first instruction onto a printable media; wherein such at least one scale and such at least one first instruction are printed onto such at least one printable media; and wherein such at least one scale is cut out from such at least one printable media prior to use.

In accordance with another preferred embodiment hereof, this invention provides a method, for allowing at least one customer to make at least one durable measuring device, comprising the steps of: such customer electronically downloading and printing instructions for measuring such customer's proper size; wherein such instructions comprise at least one printable measuring tool; wherein such instructions comprise the step of applying transparent tape to the front and back surfaces of such at least one printable measuring tool; printing such at least one printable measuring tool onto paper; applying transparent tape to the front and back surfaces of such at least one printable measuring tool; and cutting such at least one printable measuring tool out of the paper.

Even further, it provides such a method, wherein the step of printing such at least one printable measuring tool onto paper comprises the step of printing at least one printable wrist circumference measuring tool onto paper. Even further, it provides such a method, wherein the step of printing such at least one printable measuring tool onto paper comprises the step of printing at least one printable head circumference measuring tool onto paper. Even further, it provides such a method, wherein the step of printing such at least one printable measuring tool onto paper comprises the step of printing at least one printable digit circumference measuring tool onto paper. Even further, it provides such a method, wherein the step of printing such at least one printable measuring tool onto paper comprises the step of printing at least one printable neck circumference measuring tool onto paper. Even further, it provides such a method, wherein the step of printing such at least one printable measuring tool onto paper comprises the step of printing at least one watch lug width measuring tool onto paper.

In accordance with another preferred embodiment hereof, this invention provides a kit, for allowing a customer to receive a properly sized article shipped from a vendor, comprising: at least one downloadable printable wrist circumference measurer; at least one downloadable printable head circumference measurer; at least one downloadable printable digit circumference measurer; at least one downloadable printable neck circumference measurer; and at least one downloadable printable watch lug width measurer.

In accordance with another preferred embodiment hereof, this invention provides a kit, for allowing a customer to receive a properly sized article shipped from a vendor, comprising: at least one downloadable printable wrist circumference measurer; and at least one downloadable printable watch lug width measurer.

### BRIEF DESCRIPTION OF THE DRAWINGS

FIG. 1 shows a front view of a portion of a wearable-article sizing system according to a preferred embodiment of the present invention.

FIG. 2 shows a front view of a portion of a wearable-article sizing system according to a preferred embodiment of the present invention.

FIG. 3 shows diagrams illustrating several of the instructions described in FIG. 2.

FIG. 4 shows a front view of a portion of a wearable-article sizing system according to a preferred embodiment of the present invention.

FIG. 5 shows a diagram of the encircler portion of the head encircler kit, according to FIG. 4, prior to assembly.

FIG. 6 shows diagrams illustrating several of the instructions described in FIGS. 4 and 5.

FIG. 7 shows as a conversion chart and an alternate set of measuring instructions that are part of the head encircler kit according to FIG. 4.

FIG. 8 shows a front view of a portion of a wearable-article sizing system according to a preferred embodiment of the present invention.

FIG. 9 shows diagrams illustrating several of the instructions described in FIG. 8.

FIG. 10 shows a front view of a portion of a wearable-article sizing system according to a preferred embodiment of the present invention.

FIG. 11 shows diagrams illustrating several of the instructions described in FIG. 10.

FIG. 12 shows a front view of a wearable-article measuring kit, as printed onto paper.

FIG. 13 shows a front view of a watch-measuring kit, as printed onto paper.

FIG. 14 is a simplified relational diagram of preferred steps in a method of using a wearable-article sizing system according to a preferred embodiment of the present invention.

FIG. 15 is a simplified flow diagram of preferred steps in a method of utilizing a wearable-article sizing system according to a preferred embodiment of the present invention.

FIG. 16 is a simplified flow diagram of preferred steps in a method of utilizing a wearable-article sizing system according to a preferred embodiment of the present invention.

FIG. 17 is a perspective view of customer downloading a wearable-article sizing system from shopping website using a computer.

FIG. 18 is a perspective view of customer using printer to print a wrist encircler kit (with an encircler) onto paper.

FIG. 19 is a perspective view of customer cutting the encircler from the wrist encircler kit using scissors.

FIG. 20 is a perspective view of the encircler, which has been encircled around the wrist of the customer and held in place with a holder (in this example, a paper clip).

FIG. 21 is a perspective view of the customer making a mark of their wrist size on the mark line of the encircler using a pen.

FIG. 22 is a perspective view of the customer cutting the encircler at the mark using scissors.

FIG. 23 is a perspective view of the encircler being measured from the measure edge to the mark using a ruler.

FIG. 24 is an enlarged view of the ruler indicia and alignment indicia according to FIG. 1.

### DETAILED DESCRIPTION OF THE BEST MODES AND PREFERRED EMBODIMENTS OF THE INVENTION

Recently, a wide range of interactive devices has been developed to provide information to a variety of users via communications networks. These interactive devices include, for example, computers connected to various computer on-line services, interactive kiosks, interactive television systems, a variety of other wired and wireless devices, such as personal data assistants (PDAs), and the like. In particular, the popularity of computer on-line services has grown immensely in popularity over the last decade. Computer on-line services are provided by a wide variety of different companies.

In general, most computer on-line services are accessed via the Internet. The Internet is a global network of computers. One popular part of the Internet is the World Wide Web, or the “Web”. The World Wide Web contains computers that display graphical and textual information. Computers that provide information on the World Wide Web are typically called “Web sites”. A Web site is defined by an Internet address that has an associated electronic page, often called a “home page”. Generally, a home page is an electronic document that organizes the presentation of text, graphical images, audio and video into a desired display. These Web sites are operated by a wide variety of entities, which are typically called “providers”. Recently, more and more retailers are utilizing Internet websites to sell merchandise.

User 602 may access the Internet via a dedicated high-speed line or by using a personal computer (PC) equipped with a conventional modem or a variety of other wired and wireless devices. Special interface software, called “browser” software, is installed within the PC or other access device. When user 602 wishes to access the Internet by normal telephone line, for example, an attached modem is automatically instructed to dial the telephone number associated with the local Internet host server. User 602 can then access information at any address accessible over the Internet. Two well-known web browsers, for example, are the Netscape Navigator browser marketed by Netscape Communications Corporation and the Internet Explorer browser marketed by Microsoft Corporation.

Information exchanged over the Internet is typically encoded in HyperText Mark-up Language (HTML) format. The HTML format is a scripting language that is used to generate the home pages for different content providers. In this setting, a content provider is an individual or company that places information (content) on the Internet so that others can access it. As is well known in the art, the HTML format is a set of conventions for marking different portions of a document so that each portion appears in a distinctive format. For example, the HTML format identifies or “tags” portions of a document to identify different categories of text (e.g., the title, header, body text, etc.). When a web browser accesses an HTML document, the web browser reads the embedded tags in the document so it appears formatted in the specified manner.

An HTML document can also include hyperlinks, which allow user 602 to move from one document to another document on the Internet. A hyperlink is an underlined or otherwise emphasized portion of text that, when selected using an input device such as a mouse, activates a software connection module that allows user 602 to jump between documents or pages (i.e., within the same Web site or to other Web sites). Hyperlinks are well known in the art, and have been sometimes referred to as anchors. The act of selecting the hyperlink is often referred to as “clicking on” the hyperlink (also referred to as clicking on a button).

All of these technologies have provided people increased convenience at accomplishing a variety of tasks, including shopping. The present invention adds further functionality to online shopping capabilities by allowing customers to have watches (and other types of jewelry) custom-sized before they are shipped to the customer thereby provided a value-added service which can generate additional revenue and/or market share.

Glossary of General Terms and Acronyms

The following terms and acronyms are explained below as background and as used in the detailed description:

Internet. A collection of interconnected (public and/or private) networks that are linked together by a set of standard protocols to form a distributed network. While this term is intended to refer to what is now commonly known as the Internet, it is also intended to encompass variations, which may be made in the future, including changes and additions to existing standard protocols.

HyperText Markup Language (HTML). A standard coding convention and set of codes for attaching presentation and linking attributes to informational content within documents. During a document authoring stage, the HTML codes (referred to as “tags”) are embedded within the informational content of the document. When the Web document (or “HTML document”) is subsequently transferred from a Web server to a Web browser, the codes are interpreted by the Web browser and used to parse and display the document. In addition to specifying how the Web browser is to display the document, HTML tags can be used to create links to other websites and other Web documents (commonly referred to as “hyperlinks”).

HyperText Transport Protocol (HTTP). The standard World Wide Web client-server protocol used for the exchange of information (such as HTML documents, and client requests for such documents) between a Web browser and a Web server. HTTP includes a number of different types of messages that can be sent from the client to the server to request different types of server actions. For example, a “GET” message, which has the format GET, causes the server to return the document or file located at the specified Universal Resource Locator (URL).

PDA (Personal Digital Assistant). This term is sometimes used herein to refer to a small hand held computer with or without wireless access to the Internet. A lightweight, hand-held, usually pen-based computer used as a personal organizer.

Uniform Resource Locator (URL). A unique address which fully specifies the location of a file or other resource on the Internet. The general format of a URL is protocol://machine address:port/path/filename. The port specification is optional, and if none is entered by user 602, the Web browser defaults to the standard port for whatever service is specified as the protocol. For example, if HTTP is specified as the protocol, the Web browser will use the HTTP default port. The machine address in this example is the domain name for the computer or device on which the file is located.

World Wide Web (“Web”). Used herein to refer generally to both (1) a distributed collection of interlinked, user-viewable hypertext documents (commonly referred to as “Web documents”, “Web pages”, “electronic pages” or “home pages”) that are accessible via the Internet, and (2) the client and server software components that provide user access to such documents using standardized Internet protocols. Currently, the primary standard protocol for allowing applications to locate and acquire Web documents is the HyperText Transfer Protocol (HTTP), and the electronic pages are encoded using the HyperText Markup Language (HTML). However, the terms “World Wide Web” and “Web” are intended to encompass future markup languages and transport protocols that may be used in place of or in addition to the HyperText Markup Language and the HyperText Transfer Protocol.

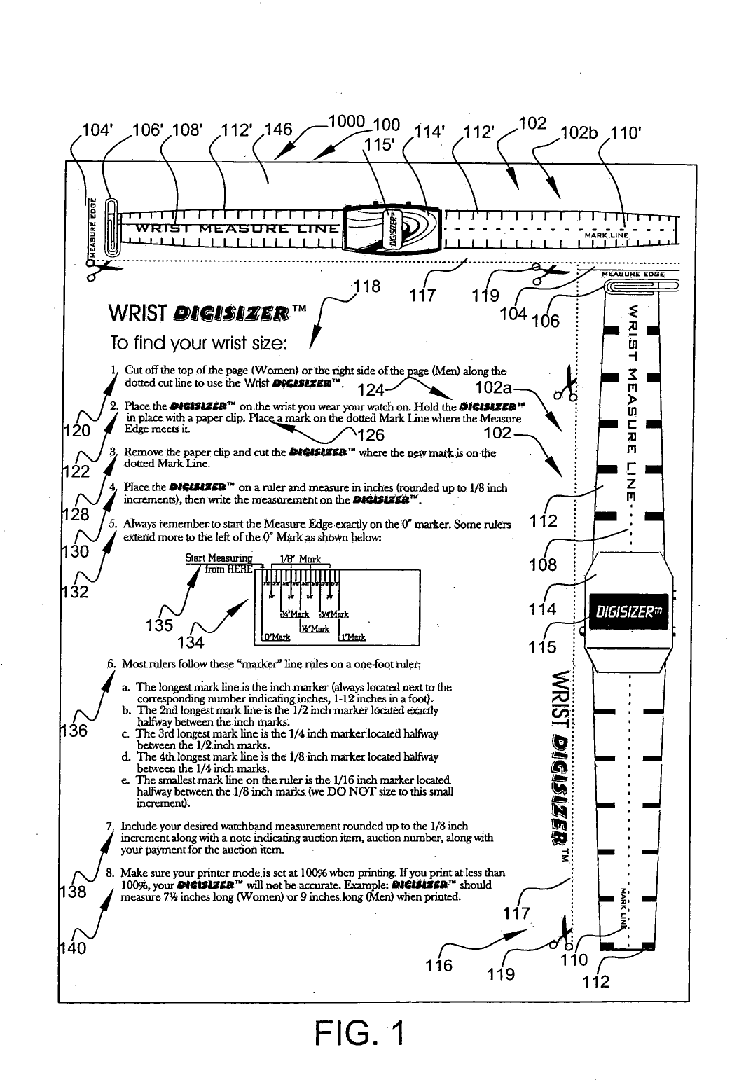

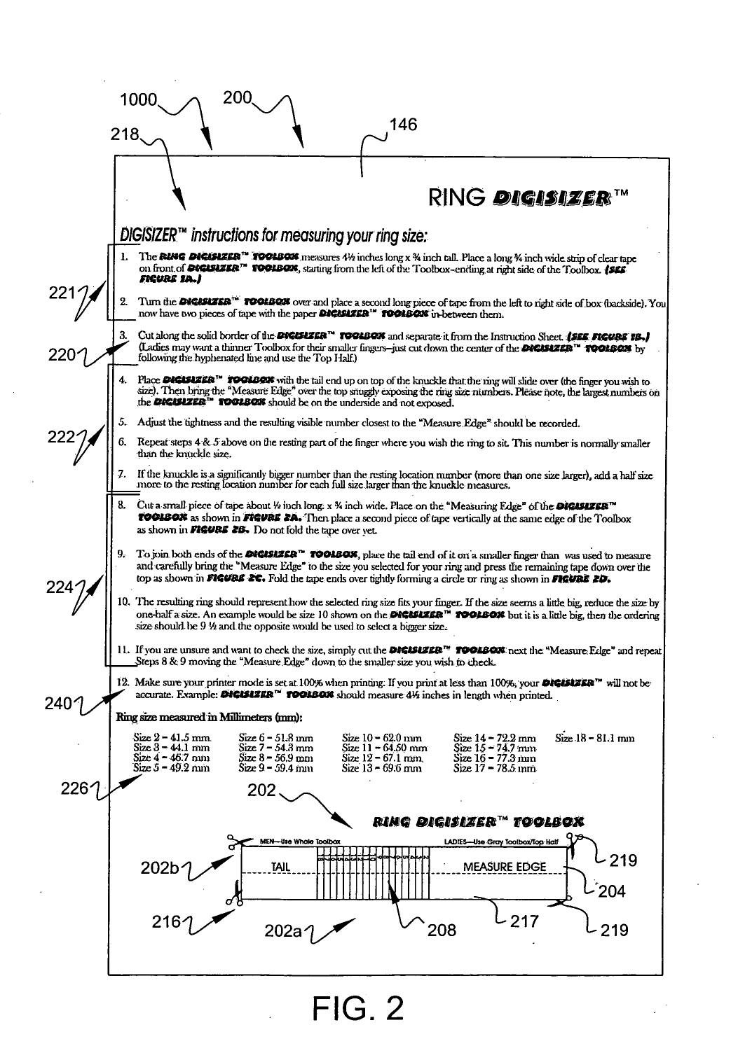

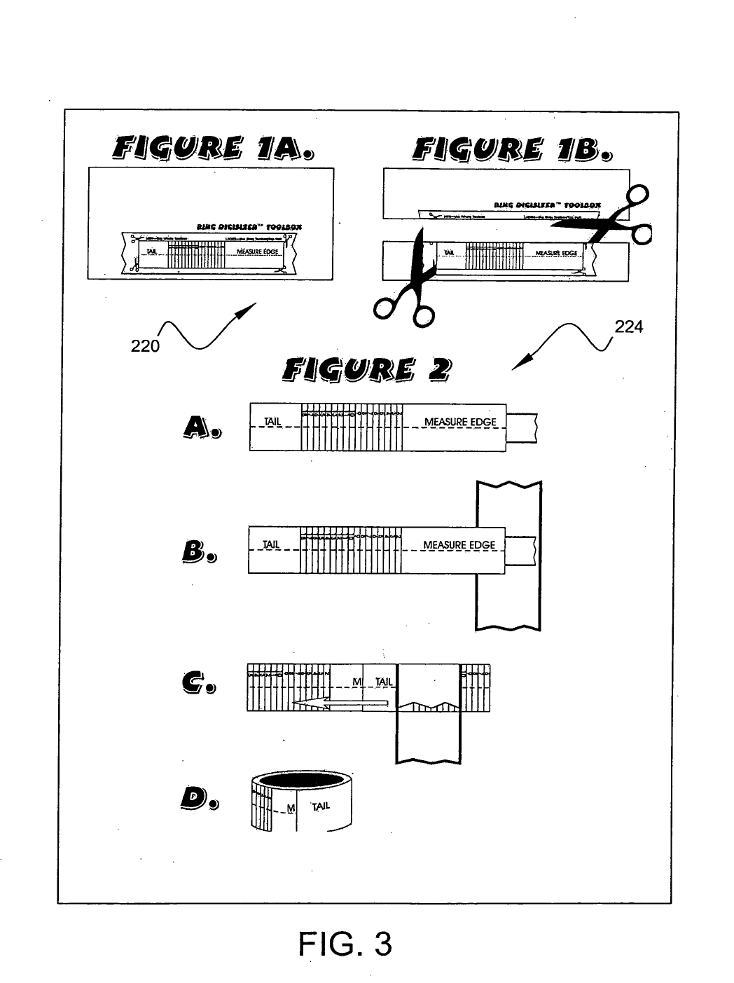

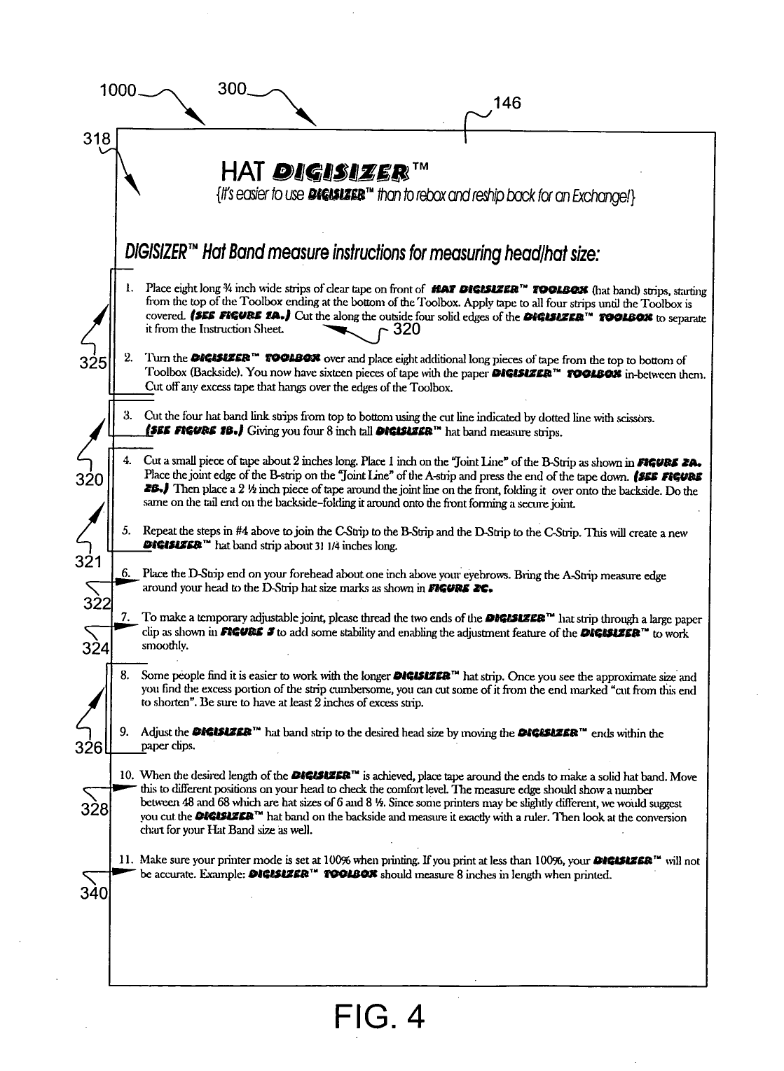

FIG. 1 shows a front view of a portion of a wearable-article sizing system 1000 according to a preferred embodiment of the present invention. Preferably, wearable-article sizing system 1000 comprises electronically downloadable and printable measuring devices for wearable-article sizing, such as, for example, watchband length, watchband width, bracelets, rings, necklaces, etc. Preferably, circumferential measurements (such as watchband length) are measured with encircling measuring devices, and straight, flat measurements (such as watchband/lug width) are measured with flat measuring devices. Preferably, the pattern for the measuring device is downloaded, is accurately printed onto paper, and the measuring device is then cut out (and assembled, if required) from the paper.

Preferably, wearable-article sizing system 1000 comprises at least one wrist encircler kit 100. Preferably, wrist encircler kit 100 comprises a sheet of paper 146 having printed thereon instructions 118 and at least one encircler 102.

Preferably, cutting indicia 116 assist in showing user 602 (also referred to as a customer 602 in this specification) preferred locations for cutting wrist encircler kit 100 in order to remove encircler 102a (and/or smaller encircler 102b) from the remainder of wrist encircler kit 100. Preferably, cutting indicia 116 comprises dotted lines 117 and scissors symbols 119, as shown. Preferably, encircler 102 becomes usable after being cut out of the paper 146, as further described below. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, etc.

Preferably, encircler 102 (at least embodying herein wherein such at least one encircler is adapted to encircle at least one wrist of the customer) comprises at least one larger encircler 102a (for use by people with larger wrists, such as, for example, men) and at least one smaller encircler 102b (for use by people with smaller wrists, such as, for example, women and children), as shown. Preferably, encircler 102 is a long, narrow strip sized to fit around the wrist of most users. Preferably, encircler 102a is about 1½ inches wide and about 9½ inches long, as printed. Preferably, smaller encircler 102b is about 1 inch wide and about 8 inches long, as printed. Upon reading the teachings of this specification, those with ordinary skill in the art will now understand that, under appropriate circumstances, considering issues such as targeted customer, type of watch or other type of article being sized, etc., other kit and encircler arrangements may suffice, such as, for example, other kit sheet materials than paper, including only a single encircler in an encircling kit, including many encirclers in an encircling kit, different widths and lengths for encirclers, etc.

Preferably, encircler 102 comprises measure edge 104, as shown. Preferably, measure edge 104 comprises a line, which is located at one end of encircler 102, as shown. Preferably, measure edge 104 is labeled with words such as, for example, “measure edge” so that users 602 can easily identify it as such. Upon reading the teachings of this specification, those with ordinary skill in the art will now understand that, under appropriate circumstances, considering issues such as language and symbols recognized by targeted customers, etc., other measure edge arrangements may suffice, such as, for example, other types of indicia, using different words to label the measure edge, omitting the words to label the measure edge, using something other than a line for the measure edge (for example, a dot), etc.

Preferably, encircler 102 comprises holder indicia 106, as shown, to show user 602 where to place holder 148 (which is not shown, but which is preferably an ordinary paper clip). Preferably, holder indicia 106 comprises a representation of a paper clip, as shown. Preferably, holder indicia 106 is located near measure edge 104, as shown. Upon reading the teachings of this specification, those with ordinary skill in the art will now understand that, under appropriate circumstances, considering issues such as type of holder to be used, language and symbols recognized by targeted customers, etc., other holder indicia arrangements may suffice, such as, for example, representations of a holder other than a paperclip, using words such as “paperclip here”, or other words, etc.

Preferably, encircler 102 comprises watch representation indicia 114, as shown. Preferably, watch representation indicia 114 appear like the face of a watch in order to provide user 602 with familiarity thereby assisting user 602 in orienting and locating encircler 102 where user 602 normally wears a watch. Preferably, watch representation indicia 114 are located near the middle of the length of encircler 102, as shown. Preferably, watch representation indicia 114 comprise encircler-branding indicia 115, as shown. Preferably, the portions of encircler 102 on each side of watch representation indicia 114 comprise wristband representation indicia 112, as shown, which appear like the wristband portions of a watch in order to provide user 602 with familiarity thereby assisting user 602 in orienting and locating encircler 102 where user 602 normally wears a watch. Upon reading the teachings of this specification, those with ordinary skill in the art will now understand that, under appropriate circumstances, considering issues such as target customers, type of jewelry being sized, etc., other watch representation indicia arrangements may suffice, such as, for example, representations of different types of watches (such as, an analog watch), etc.

Preferably, encircler 102 comprises measure line indicia 108 and mark line indicia 110, as shown. Preferably, measure line indicia 108 comprise a dashed line and words, such as, for example, “wrist measure line”, as shown. Preferably, mark line indicia 110 comprise a dashed line and words such as, for example, “mark line”, as shown. Preferably, measure line indicia 108 are differentiated from the mark line indicia 110 in appearance to assist user 602 in understanding how to orient and use encircler 102 to measure the wrist of user 602. Preferably, measure line indicia 108 are located on the portion of encircler 102 between watch representation indicia 114 and measure edge 104, as shown. Preferably, mark line indicia 110 are located on the portion of encircler 102 on the other side of watch representation indicia 114 opposite measure edge 104, as shown. Upon reading the teachings of this specification, those with ordinary skill in the art will now understand that, under appropriate circumstances, considering issues such as the instructions for use of the encircler, etc., other measure line indicia and mark line indicia arrangements may suffice, such as, for example, using different words, using different kinds of dashed, dotted, or solid lines, etc., omitting measure line indicia and/or mark line indicia, etc. Upon reading the teachings of this specification, those of ordinary skill in the art will now understand that, under appropriate circumstances, such as user preference, advances in technology, intended use, etc., other indicia, such as accurate measuring indicia, an entire printed ruler, measurement indicia on the watchband, additional cutting lines, etc., may suffice.

Preferably, smaller encircler 102b comprises the same features as encircler 102, as shown, (such as, for example, measure edge 104′ analogous to measure edge 104, holder indicia 106′ analogous to holder indicia 106, measure line indicia 108′ analogous to measure line indicia 108, mark line indicia 110′ analogous to mark line indicia 110, wristband representation indicia 112′ analogous to wristband representation indicia 112, watch representation indicia 114′ analogous to watch representation indicia 114, encircler branding indicia 115′ analogous to encircler branding indicia 115, etc.).

Preferably, wrist encircler kit 100 comprises instructions 118. Preferably, instructions 118 comprise encircler removal instructions 120. Preferably, encircler removal instructions 120 instruct user 602 how to remove encircler 102, preferably larger encircler 102a and/or smaller encircler 102b, from wrist encircler kit 100. Preferably, encircler removal instructions 120 instruct user 602 to cut wrist encircler kit 100. Preferably, encircler removal instructions 120 comprise words such as, for example, “Cut off the top of the page (Women) or the right side of the page (Men) along the dotted cut line to use the Wrist Digisizer™.”, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, different words, different instructions, etc.

Preferably, instructions 118 comprise encircling instructions 122. Preferably, encircling instructions 122 instruct user 602 how and where to place encircler 102 (preferably, larger encircler 102a or smaller encircler 102b). Preferably, encircling instructions 122 instruct user 602 to encircle their wrist with encircler 102 (preferably, larger encircler 102a or smaller encircler 102b) where user 602 will wear their watch. Preferably, encircling instructions 122 comprise words such as, for example, “Place the Digisizer™ on the wrist you wear your watch on.”, as shown. Upon reading the teachings of this specification, those with ordinary skill in the art will now understand that, under appropriate circumstances, considering issues such as the language of targeted customers, etc., other encircling instruction arrangements may suffice, such as, for example, using different words, providing a diagram, etc.

Preferably, instructions 118 comprise holder instructions 124. Preferably, holder instructions 124 instruct user 602 how to hold encircler 102 (preferably, larger encircler 102a or smaller encircler 102b) in place on the wrist of user 602. Preferably, holder instructions 124 instruct user 602 to hold encircler 102 (preferably, larger encircler 102a or smaller encircler 102b) in place with a holder 148 (not shown in FIG. 1), which is, preferably, a paper clip. Preferably, holder instructions 124 comprise words such as, for example, “Hold the Digisizer™ in place with a paper clip.”, as shown. Upon reading the teachings of this specification, those with ordinary skill in the art will now understand that, under appropriate circumstances, considering issues such as the language of targeted customers, etc., other holder instruction arrangements may suffice, such as, for example, using different words, providing a diagram, etc.

Preferably, instructions 118 comprise marking instructions 126. Preferably, marking instructions 126 instruct user 602 how and where to mark encircler 102 (preferably, larger encircler 102a or smaller encircler 102b) in order to assist in measuring the wrist size of user 602. Preferably, marking instructions 126 instruct user 602 to make a mark 150 (not shown in FIG. 1) on the mark line indicia 110 of encircler 102 (preferably, larger encircler 102a or smaller encircler 102b) where the measure edge 104 crosses the mark line indicia 110. Preferably, marking instructions 126 comprise words, such as, for example, “Place a mark on the dotted Mark Line where the Measure Edge meets it.”, as shown. Upon reading the teachings of this specification, those with ordinary skill in the art will now understand that, under appropriate circumstances, considering issues such as the language of targeted customers, etc., other marking instruction arrangements may suffice, such as, for example, using different words, providing a diagram, etc.

Preferably, instructions 118 comprise cutting instructions 128. Preferably, cutting instructions 128 instruct user 602 to remove holder 148 (not shown in FIG. 1), and cut encircler 102 (preferably, larger encircler 102a or smaller encircler 102b) at the location of mark 150, perpendicular to mark line indicia 110 (not shown in FIG. 1). Preferably, cutting instructions 128 comprise words such as, for example, “Remove the paper clip and cut the Digisizer™ where the new mark is on the dotted Mark Line.”, as shown. Upon reading the teachings of this specification, those with ordinary skill in the art will now understand that, under appropriate circumstances, considering issues such as the language of targeted customers, etc., other cutting instruction arrangements may suffice, such as, for example, using different words, providing a diagram, etc.

Preferably, instructions 118 comprise measuring instructions 130. Preferably, measuring instructions 130 instruct user 602 how to measure encircler 102 (preferably, larger encircler 102a or smaller encircler 102b) in order to determine the wrist size of user 602. Preferably, measuring instructions 130 instruct user 602 to measure the length of encircler 102 (preferably, larger encircler 102a or smaller encircler 102b) from the measure edge 104 to the mark 150 (where encircler 102 has preferably been cut) using a linear measuring tool, preferably using a ruler 152, as shown and described herein. Preferably, measuring instructions 130 comprise words such as, for example, “Place the Digisizer™ on a ruler and measure in inches (rounded up to 1/8 inch increments), then write the measurement on the Digisizer™.”, as shown. Upon reading the teachings of this specification, those with ordinary skill in the art will now understand that, under appropriate circumstances, considering issues such as the language of targeted customers, etc., other measuring instruction arrangements (to obtain similar goals) may suffice, such as, for example, using different words, providing a diagram, etc.

Preferably, instructions 118 comprise ruler instructions 132. Preferably, ruler instructions 132 (at least embodying herein wherein such first instruction means comprises ruler instruction means for instructing the customer in the use of a ruler to measure a length of such encircler means; and at least embodying herein wherein such at least one instruction comprises at least one ruler instruction adapted to instruct the customer in the use of a ruler to measure a length of such at least one encircler) instruct user 602 how to interpret and use a ruler 152. Preferably, ruler instructions 132 instruct user 602 to align measure edge 104 with the zero mark (0″ marker) on ruler 152. Preferably, ruler instructions 132 comprise words such as, for example, “Always remember to start the Measure Edge exactly on the 0″ marker. Some rulers extend more to the left of the 0″ Mark as shown below:”, as shown. Upon reading the teachings of this specification, those with ordinary skill in the art will now understand that, under appropriate circumstances, considering issues such as the language of targeted customers, etc., other ruler instruction arrangements (to obtain similar goals) may suffice, such as, for example, using different words, etc.

Preferably, instructions 118 comprise ruler indicia 134. Preferably, ruler indicia 134 (at least embodying herein wherein such ruler instruction means comprises ruler indicia means for visually illustrating the use of a ruler to measure a length of such encircler means; and at least embodying herein at least one ruler indicia adapted to visually illustrate such at least one ruler instructions) show a sample portion of a ruler 152 labeled so that user 602 can easily read and interpret it. Preferably, ruler indicia 134 comprise alignment indicia 135 that shows user 602 where to align the measure edge 104 with the zero mark (0″ marker) on the sample portion of ruler 152, as shown. Preferably, alignment indicia 135 comprise words such as, for example, “Start measuring from HERE” and an arrow pointing to the 0″ mark on the sample portion of ruler 152, as shown. Upon reading the teachings of this specification, those with ordinary skill in the art will now understand that, under appropriate circumstances, considering issues such as the language of targeted customers, etc., other ruler indicia and alignment indicia arrangements (to obtain similar goals) may suffice, such as, for example, using different words, using different diagrams, describing a metric ruler, etc.

Preferably, instructions 118 comprise marker line rules 136. Preferably, marker line rules 136 instruct user 602 how to understand and read the different marker lines on a typical ruler 152. Preferably, marker line rules 136 comprise words such as, for example, “Most rulers follow these ‘marker’ line rules on a one-foot ruler: a) The longest mark line is the inch marker (always located next to the corresponding number indicating inches, 1-12 inches in a foot); b) The 2nd longest mark line is the 1/2 inch marker located exactly halfway between the inch marks; c) The 3rd longest mark line is the 1/4 inch marker located halfway between the 1/2 inch marks; d) The 4th longest mark line is the 1/8 inch marker located halfway between the 1/4 inch marks; 4) The smallest mark line on the ruler is the 1/16 inch marker located halfway between the 1/8 inch marks (we DO NOT size to this small increment).”, as shown. Upon reading the teachings of this specification, those with ordinary skill in the art will now understand that, under appropriate circumstances, considering issues such as the language of targeted customers, etc., other marker line rules arrangements (to obtain similar goals) may suffice, such as, for example, using different words, describing a metric ruler, etc.

Preferably, instructions 118 comprise requisition instructions 138. Preferably, requisition instructions 138 instruct user 602 which information to include in a request for watchband sizing. Preferably, requisition instructions 138 comprise words such as, for example, “Include your desired watchband measurement rounded up to the 1/8 inch increment along with a note indicating auction item, auction number, along with your payment for the auction item.”, as shown. Upon reading the teachings of this specification, those with ordinary skill in the art will now understand that, under appropriate circumstances, considering issues such as the language of targeted customers, whether the watch (or other wearable article) was bought as the result of a winning bid at an auction or directly purchased from a retailer, etc., other requisition instruction arrangements may suffice, such as, for example, using different words, requiring different documentation items, etc.

Preferably, instructions 118 comprise printing instructions 140. Preferably, printing instructions 140 instruct user 602 how to print wrist encircler kit 100 so that encircler 102 is exactly the correct size. Preferably, printing instructions 140 comprise words such as, for example, “Make sure your printer mode is set at 100% when printing. If you print at less than 100%, your DIGISIZER™ will not be accurate. Example: DIGISIZER™ should measure 7½ inches long (Women) or 9 inches long (Men) when printed.”. Upon reading the teachings of this specification, those with ordinary skill in the art will now understand that, under appropriate circumstances, considering issues such as the language of targeted customers, data format, etc., other printing instructions (to obtain similar goals) may suffice, such as, for example, using different words, using other methods to verify the size, etc. Upon reading the teachings of this specification, those of ordinary skill in the art will now understand that, under appropriate circumstances, such as user preference, advances in technology, intended use, etc., other instructions, such as lamination instructions, assembly instructions, etc., may suffice.

Preferably, encircler 102 comprises bendable/flexible material so that encircler 102 can be wrapped around the wrist of user 602, and also so that encircler 102 (at least embodying herein wherein such at least one encircler is adapted to be straightened) can be straightened (flattened) out so that it can be conveniently measured by a linear measurement tool, such as, for example, a ruler 152 (at least embodying herein wherein such at least one encircler may be measured by a linear measuring tool). Preferably, encircler 102 (at least embodying herein at least one encircler to encircle a wrist of the customer) does not comprise stretchable material, so that changes in the length of encircler 102 are negligible in order to insure accurate length measurements. Preferably, wrist encircler kit 100 comprising encircler 102 is printed onto paper 146, preferably at least one piece of 8½″×11″ paper. Upon reading the teachings of this specification, those with ordinary skill in the art will now understand that, under appropriate circumstances, considering issues such as improvements in print media, and alternatives to paper for printing, etc., other materials may suffice, such as, for example, using film, cloth, synthetic materials, other paper sizes, etc.

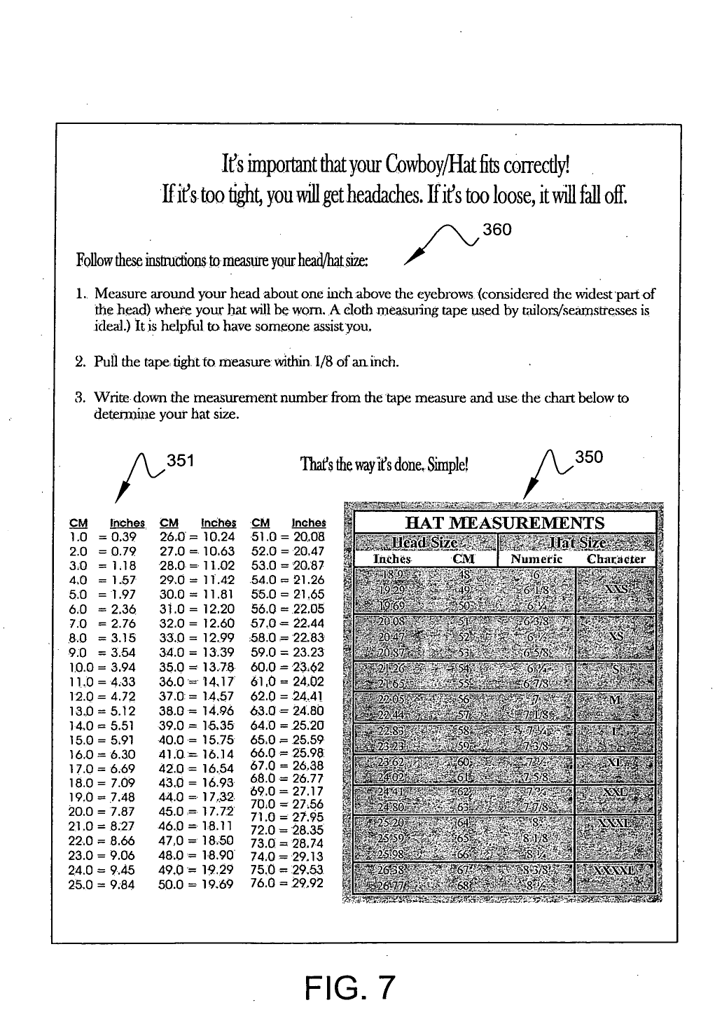

Preferably, wrist encircler kit 100 can be printed by a customer by accessing and downloading the data from an Internet website. Preferably, user 602 prints the wrist encircler kit 100 on the printer 158 of user 602 (at least embodying herein the step of printing at least one printable wrist circumference measuring tool onto paper) (as shown in FIG. 7), preferably onto paper 146 (at least embodying herein wherein such at least one encircler may be measured by a linear measuring tool). Upon reading the teachings of this specification, those of ordinary skill in the art will now understand that, under appropriate circumstances, such as user preference, advances in technology, intended use, etc., various file formats, such as an Adobe Acrobat™ .PDF file, a word processing document, a computerized drawing, an image file, etc., may suffice.

FIG. 2 shows a front view of a portion of a wearable-article sizing system 1000 according to a preferred embodiment of the present invention. Preferably, wearable-article sizing system 1000 comprises at least one finger encircler kit 200 (which is equally useful for measuring toes, or any other human digit).

Preferably, finger encircler kit 200 comprises a sheet of paper 146 having printed thereon instructions 218 (at least embodying herein first instruction means for instructing the at least one customer in the use of such encircler means to provide a measurement for the wearable article, and at least embodying herein at least one first instruction adapted to instruct a customer in the use of such at least one encircler to provide a measurement for the wearable article) and at least one encircler 202. Preferably, encircler 202 comprises at least one wider encircler 202a (for use by people with larger fingers and/or toes, such as, for example, men) and at least one narrower encircler 202b (for use by people with smaller fingers and/or toes, such as, for example, women and children), as shown. Preferably, encircler 202 (at least embodying herein encircler means for encircling at least one article-wearable body part of the at least one customer, and at least embodying herein at least one encircler adapted to encircle an article-wearable body part of the customer) is a long, narrow strip sized to fit around the finger of most users. Preferably, wider encircler 202a is about 3/4 inches wide and about 4½ inches long, as printed. Preferably, narrower encircler 202b is about 3/8 inches wide and about 4½ inches long, as printed. Upon reading the teachings of this specification, those with ordinary skill in the art will now understand that, under appropriate circumstances, considering issues such as targeted customer, type of ring or other type of article being sized, etc., other kit and encircler arrangements may suffice, such as, for example, other kit sheet materials than paper, including only a single encircler in an encircling kit, including many encirclers in an encircling kit, different widths and lengths for encirclers, etc.

Preferably, finger encircler kit 200 comprises cutting indicia 216, as shown, to assist in showing user 602 preferred locations for cutting finger encircler kit 200 in order to remove encircler 202a (and/or smaller encircler 202b) from the remainder of finger encircler kit 200. Preferably, cutting indicia 216 comprise solid lines 217 and scissors symbols 219, as shown. Preferably, encircler 202 becomes usable after being cut out of the paper 146. Upon reading the teachings of this specification, those with ordinary skill in the art will now understand that, under appropriate circumstances, considering issues such as language and symbols recognized by targeted customers, etc., other cutting indicia arrangements may suffice, such as, for example, dotted and/or dashed lines, omitting scissors symbols, etc.

Preferably, encircler 202 comprises measure edge 204, as shown. Preferably, measure edge 204 comprises a line, which is located at one end of encircler 202, as shown. Preferably, measure edge 204 is labeled with the words “measure edge” so that users can easily identify it as such. Upon reading the teachings of this specification, those with ordinary skill in the art will now understand that, under appropriate circumstances, considering issues such as language and symbols recognized by targeted customers, etc., other measure edge arrangements may suffice, such as, for example, other types of indicia, using different words to label the measure edge, omitting the words to label the measure edge, using something other than a line for the measure edge (for example, a dot), etc.

Preferably, encircler 202 comprises ring size gauge 208. Preferably, ring size gauge 208 marks standard ring size circumferences, as measured from the measure edge 204. Preferably, a wide range of men's and women's ring sizes is provided. Most preferably, ring sizes for fingers and toes between about size 2 (about 41.5 mm) and about size 18 (about 81.1 mm) are provided. Upon reading the teachings of this specification, those with ordinary skill in the art will now understand that, under appropriate circumstances, considering issues such as the language of targeted customers, etc., other ring size indicia may suffice, such as, for example, using different words, using metric measurement, using English measurement, providing more or fewer size increments, etc.

Preferably, instructions 218 comprise lamination instructions 221. Preferably, lamination instructions 221 instruct user 602 to cover both sides (surfaces) of encircler 202 with a laminating material (such as, for example, clear adhesive tape) in order to provide strength and ease of handling to encircler 202 in use. Preferably, lamination instructions 221 comprise words such as, for example, “Place a long 3/4 inch wide strip of clear tape on the front of the Digisizer™ Toolbox, starting from the left of the Toolbox-ending at right side of the Toolbox. Turn the Digisizer™ Toolbox over and place a second long piece of tape from the left to right side of the box (backside). You now have two pieces of tape with the paper Digisizer™ Toolbox in-between them.” (at least embodying herein applying transparent tape to the front and back surfaces of such at least one printable measuring tool), as shown. Upon reading the teachings of this specification, those with ordinary skill in the art will now understand that, under appropriate circumstances, considering issues such as the language of targeted customers, etc., other lamination instruction arrangements may suffice, such as, for example, using different words, omitting the lamination instructions, etc.

Preferably, instructions 218 comprise encircler removal instructions 220. Preferably, encircler removal instructions 220 instruct user 602 how to remove encircler 202, preferably wider encircler 202a and/or narrower encircler 202b, from finger encircler kit 200. Preferably, encircler removal instructions 220 instruct user 602 to cut finger encircler kit 200. Preferably, encircler removal instructions 220 comprise words such as, for example, “Cut along the solid border of the Digisizer™ Toolbox and separate it from the Instruction Sheet. (Ladies may want a thinner Toolbox for their smaller fingers—just cut down the center of the Digisizer™ Toolbox by following the hyphenated line and use the Top Half.)”, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, different words, etc.

Preferably, instructions 218 comprise encircling instructions 222. Preferably, encircling instructions 222 instruct user 602 how and where to place encircler 202 (at least embodying herein wherein such at least one encircler is adapted to encircle at least one digit of the customer) (preferably, wider encircler 202a or narrower encircler 202b). Preferably, encircling instructions 222 (at least embodying herein at least one first instruction adapted to instruct the customer in the use of such at least one encircler) instruct user 602 to encircle and measure their finger with encircler 202 (preferably, wider encircler 202a or narrower encircler 202b) first on the knuckle, and then on the point where the ring will be worn, recording both measurements. Preferably, encircling instructions 222 comprise words such as, for example, such as, for example, “Place Digisizer™ Toolbox with the tail end up on top of the knuckle that the ring will slide over (the finger you wish to size). Then bring the “Measure Edge” over the top snuggly exposing the ring size numbers. Please note, the largest numbers on the Digisizer™ Toolbox should be on the underside and not exposed. Adjust the tightness and the resulting visible number closest to the “Measure Edge” should be recorded. Repeat the steps above on the resting part of the finger where you wish the ring to sit. This number is normally smaller than the knuckle size. If the knuckle is a significantly bigger number than the resting location number, add a half size more to the resting location number for each full size larger that the knuckle measures.”, as shown. Upon reading the teachings of this specification, those with ordinary skill in the art will now understand that, under appropriate circumstances, considering issues such as the language of targeted customers, etc., other encircling instruction arrangements may suffice, such as, for example, using different words, providing a diagram, etc.

Preferably, instructions 218 comprise holder instructions 224. Preferably, holder instructions 224 instruct user 602 how to hold encircler 202 (preferably, larger encircler 202a or smaller encircler 202b) in the shape of a ring for final fitting. Preferably, holder instructions 224 instruct user 602 to hold encircler 202 (preferably, larger encircler 202a or smaller encircler 202b) in place with a holder 248 (at least embodying herein wherein said at least one holder comprises at least one piece of adhesive tape), which is preferably a piece of tape. Preferably, holder instructions 224 comprise words such as, for example, “Cut a small piece of tape about 1/2 inch long×3/4 inch wide. Place on the “Measuring Edge” of the Digisizer™ Toolbox as shown. Then place a second piece of tape vertically at the same edge of the Toolbox as shown. Do not fold the tape over yet. To join both ends of the Digisizer™ Toolbox, place the tail end of it on a smaller finger than was used to measure and carefully bring the “Measure Edge” to the size you selected for your ring and press the remaining tape down over the top as shown. Fold the tape ends over tightly forming a circle or ring as shown. The resulting ring should represent how the selected ring size fits your finger. If the size seems a little big, reduce the size by one-half a size. An example would be size 10 shown on the Digisizer™ Toolbox but it is a little big, then the ordering size should be 9½ and the opposite would be used to select a bigger size. If you are unsure and want to check the size, simply cut the next the “Measure Edge” and repeat the taping steps moving the “Measure Edge” down to the smaller size you wish to check.”, as shown. Upon reading the teachings of this specification, those with ordinary skill in the art will now understand that, under appropriate circumstances, considering issues such as the language of targeted customers, etc., other holder instruction arrangements may suffice, such as, for example, using different words, providing a diagram, etc.

Preferably, instructions 218 comprise printing instructions 240. Preferably, printing instructions 240 instruct user 602 how to print finger encircler kit 200 so that encircler 202 is exactly the correct size. Preferably, printing instructions 240 comprise words such as, for example, “Make sure your printer mode is set at 100% when printing. If you print at less than 100%, your DIGISIZER™ will not be accurate. Example: DIGISIZER™ Toolbox should measure 4½ inches long when printed.”. Upon reading the teachings of this specification, those with ordinary skill in the art will now understand that, under appropriate circumstances, considering issues such as the language of targeted customers, data format, etc., other printing instructions (to obtain similar goals) may suffice, such as, for example, using different words, providing alternate size checking means, etc.

Preferably, instructions 218 comprise conversion chart 226. Preferably, conversion chart 226 provides conversions between the measurement units shown on encircler 202 and another unit of measurement. Preferably, conversion chart 226 converts between ring sizes and millimeters, as shown. Upon reading the teachings of this specification, those with ordinary skill in the art will now understand that, under appropriate circumstances, considering issues such as the language of targeted customers, local units of measurement, etc., other conversion charts (to obtain similar goals) may suffice, such as, for example, using different words, converting ring sizes to English measurements, etc.

Preferably, encircler 202 comprises bendable/flexible material so that encircler 202 can be wrapped around the finger of user 602. Preferably, encircler 202 does not comprise stretchable material, so that changes in length of encircler 202 (at least embodying herein at least one encircler to encircle a digit of the customer) are negligible in order to insure accurate length measurement. Preferably, encircler 202 is printed onto paper 146, preferably 8½″×11″ paper. Upon reading the teachings of this specification, those with ordinary skill in the art will now understand that, under appropriate circumstances, considering issues such as improvements in print media, and alternatives to paper for printing, etc., other materials may suffice, such as, for example, using film, cloth, synthetic materials, etc.

Preferably, finger encircler kit 200 can be printed by a customer by accessing and downloading the data from an Internet website. Preferably, user 602 prints the finger encircler kit 200 on the printer 158 of user 602 (at least embodying herein the step of printing at least one printable digit circumference measuring tool onto paper) (as shown in FIG. 7), preferably onto paper 146. Upon reading the teachings of this specification, those of ordinary skill in the art will now understand that, under appropriate circumstances, such as user preference, advances in technology, intended use, etc., various file formats, such as an Adobe Acrobat™ .PDF file, a word processing document, a computerized drawing, an image file, an automated fax, etc., may suffice. Upon reading the teachings of this specification, those of ordinary skill in the art will now understand that, under appropriate circumstances, such as user preference, advances in technology, intended use, etc., other electronic media, such as fax, Internet-capable telephones, etc., may suffice.

FIG. 3 shows diagrams of several of the instructions described in FIG. 2. In particular, encircler removal instructions 220 (at least embodying herein at least one indicium adapted to visually assist the customer to use such at least one encircler) and holder instructions 224 are illustrated.

FIG. 4 shows a front view of a portion of a wearable-article sizing system 1000 according to a preferred embodiment of the present invention. Preferably, wearable-article sizing system 1000 comprises at least one head encircler kit 300.

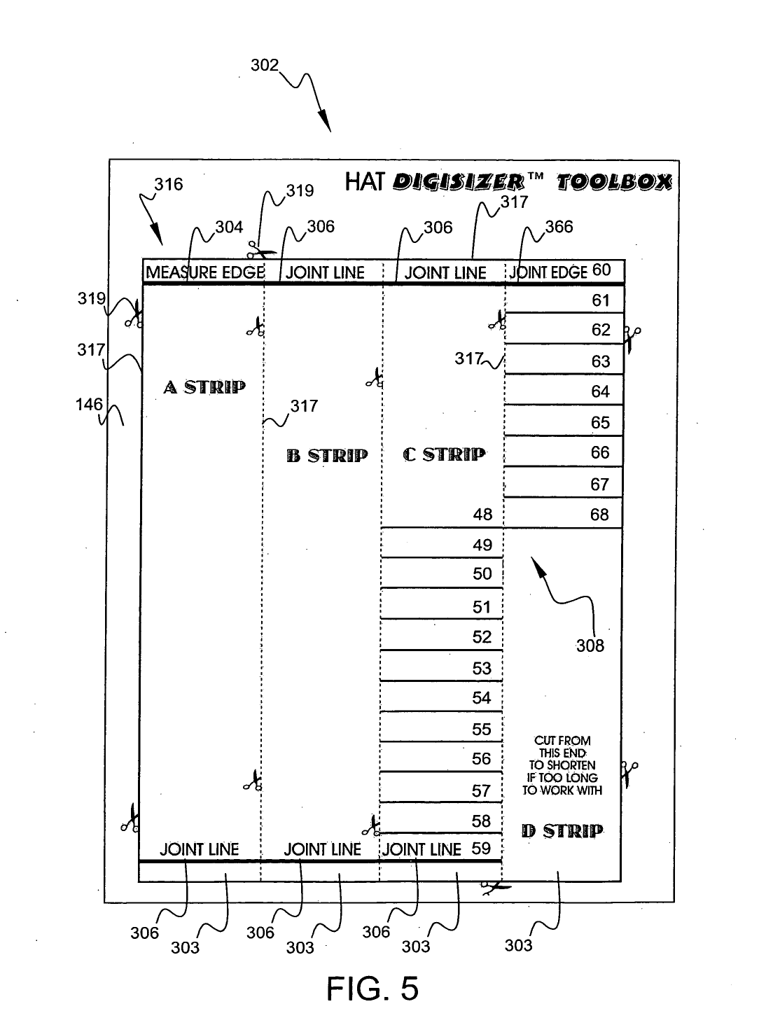

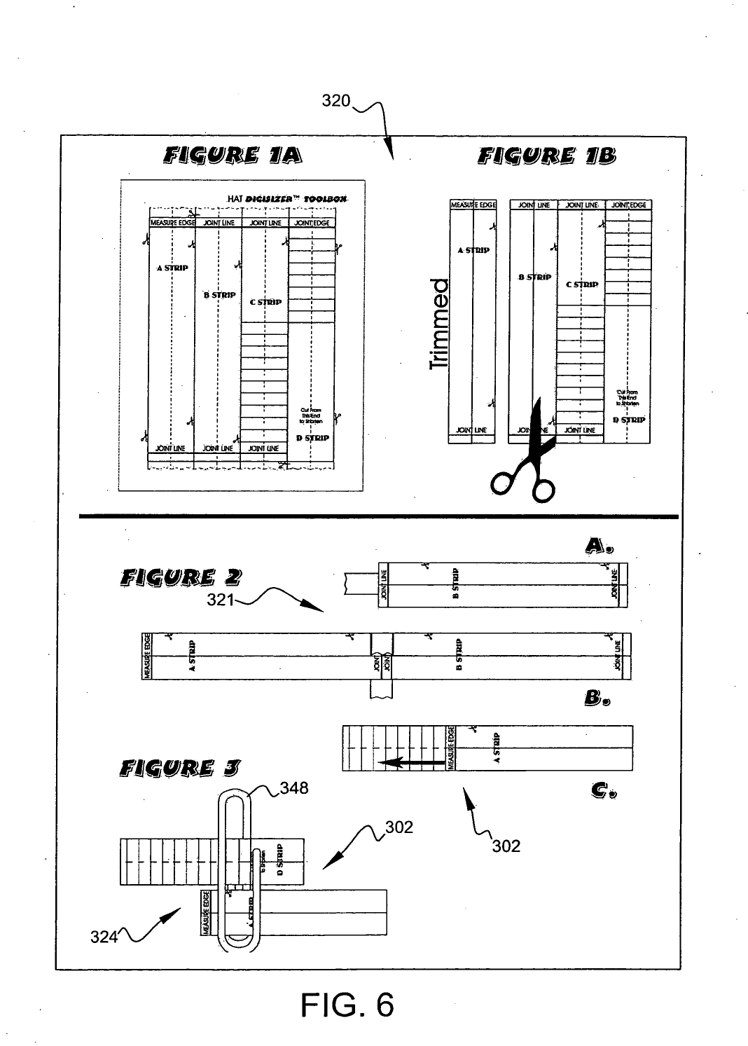

Preferably, head encircler kit 300 comprises at least one sheet of paper 146 having printed thereon instructions 318 and at least one encircler 302. Preferably, encircler 302 (at least embodying herein at least one encircler to encircle the head of the customer) is a long, narrow strip sized to fit around the head of most users. Preferably, encircler 302 (when assembled) is about 31¼ inches long and about 1¼ inches wide. Preferably, encircler 302 is assembled from four strips 303 that are each about 8 inches long and about 1¼ inches wide (as shown especially in FIG. 5 and FIG. 6). Upon reading the teachings of this specification, those with ordinary skill in the art will now understand that, under appropriate circumstances, considering issues such as targeted customer, type of hat or other type of article being sized, etc., other kit and encircler arrangements may suffice, such as, for example, other kit sheet materials than paper, including only a single encircler in an encircling kit, including many encirclers in an encircling kit, different widths and lengths for encirclers, etc.

Preferably, instructions 318 comprise lamination instructions 325. Preferably, lamination instructions 325 (at least embodying herein wherein such instructions comprise the step of applying transparent tape to the front and back surfaces of such at least one printable measuring tool) instruct user 602 to cover both sides (surfaces) of encircler 302 (at least embodying herein wherein such instructions comprise at least one printable measuring tool) with a laminating material (such as, for example, clear adhesive tape) in order to provide strength and ease of handling to encircler 302 in use. Preferably, lamination instructions 325 comprise words such as, for example, “Place eight long ¾ inch wide strips of clear tape on front of HAT DIGISIZER™ TOOLBOX (hat band) strips, starting from the top of the Toolbox ending at the bottom of the Toolbox. Apply tape to all four strips until the Toolbox is covered. Cut the along the outside four solid edges of the DIGISIZER™ TOOLBOX to separate it from the Instruction Sheet. Turn the DIGISIZER™ TOOLBOX over and place eight additional long pieces of tape from the top to bottom of the Toolbox (Backside). You now have sixteen pieces of tape with the paper DIGISIZER™ TOOLBOX in-between them. Cut off any excess tape that hangs over the edges of the Toolbox.”, as shown. Upon reading the teachings of this specification, those with ordinary skill in the art will now understand that, under appropriate circumstances, considering issues such as the language of targeted customers, etc., other lamination instruction arrangements may suffice, such as, for example, using different words, etc.

Preferably, instructions 318 comprise encircler removal instructions 320. Preferably, encircler removal instructions 320 instruct user 602 how to remove encircler 302 from head encircler kit 300. Preferably, encircler removal instructions 320 instruct user 602 to cut head encircler kit 300. Preferably, encircler removal instructions 320 comprise words such as, for example, “Cut the along the outside four solid edges of the Digisizer™ Toolbox to separate it from the Instruction Sheet. Cut the four hatband link strips from top to bottom using the cut line indicated by dotted line with scissors.”, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, different words, etc.

Preferably, instructions 318 comprise assembly instructions 321. Preferably, assembly instructions 321 instruct user 602 how to assemble the four strips 303 into one encircler 302. Preferably, assembly instructions 321 comprise words such as, for example, “Cut a small piece of tape about 2 inches long. Place 1 inch on the “Joint Line” of the B-Strip as shown. Place the joint edge of the B-strip on the “Joint Line” of the A-strip and press the end of the tape down. Then place a 2½-inch piece of tape around the joint line on the front, folding it over onto the backside. Do the same on the tail end on the backside—folding it around onto the front forming a secure joint. Repeat the steps above to join the C-Strip to the B-Strip and the D-Strip to the C-Strip. This will create a new hat band strip about 31¼ inches long.”, as shown. Preferably, encircler 302 becomes usable after being cut out of the paper 146 and after being assembled. Upon reading the teachings of this specification, those with ordinary skill in the art will now understand that, under appropriate circumstances, considering issues such as language and symbols recognized by targeted customers, etc., other assembly instructions may suffice, such as, for example, different words, providing a diagram, etc.

Preferably, instructions 318 comprise encircling instructions 322. Preferably, encircling instructions 322 instruct user 602 how and where to place encircler 302 (at least embodying herein wherein such at least one encircler is adapted to encircle the head of the customer) to measure hat size. Preferably, encircling instructions 322 instruct user 602 to encircle their head with encircler 302 where user 602 will wear their hat. Preferably, encircling instructions 322 comprise words such as, for example, “Place the D-Strip end on your forehead about one inch above your eyebrows. Bring the A-Strip measure edge around your head to the D-Strip hat size marks, as shown.”, as shown. Upon reading the teachings of this specification, those with ordinary skill in the art will now understand that, under appropriate circumstances, considering issues such as the language of targeted customers, etc., other encircling instruction arrangements may suffice, such as, for example, using different words, providing a diagram, etc.

Preferably, instructions 318 comprise holder instructions 324. Preferably, holder instructions 324 instruct user 602 how to hold encircler 302 in place on the head of user 602. Preferably, holder instructions 324 instruct user 602 to hold encircler 302 in place with a holder 348, which is preferably a paper clip. Preferably, holder instructions 324 comprise words such as, for example, “To make a temporary adjustable joint, please thread the two ends of the hat strip through a large paper clip as shown to add some stability and enable the adjustment feature of the Digisizer™ Toolbox to work smoothly.”, as shown. This instruction is diagrammed in FIG. 6. Upon reading the teachings of this specification, those with ordinary skill in the art will now understand that, under appropriate circumstances, considering issues such as the language of targeted customers, etc., other holder instruction arrangements may suffice, such as, for example, using different words, etc.

Preferably, instructions 318 comprise fitting instructions 326. Preferably, fitting instructions 326 instruct user 602 to properly fit encircler 302 around the head. Preferably, fitting instructions 326 comprise words such as, for example, “Some people find it is easier to work with the longer DIGISIZER™ hat strip. Once you see the approximate size and you find the excess portion of the strip cumbersome, you can cut some of it from the end marked “cut from this end to shorten. Be sure to have at least 2 inches of excess strip. Adjust the DIGISIZER™ hat band strip to the desired head size by moving the DIGISIZER™ ends within the paper clips. When the desired length of the DIGISIZER™ is achieved, place tape around the ends to make a solid hat band. Move this to different positions on your head to check the comfort level. The measure edge should show a number between 48 and 68 which are hat sizes of 6 and 8½.”, as shown. Upon reading the teachings of this specification, those with ordinary skill in the art will now understand that, under appropriate circumstances, considering issues such as the language of targeted customers, etc., other cutting instruction arrangements may suffice, such as, for example, using different words, etc.

Preferably, instructions 318 comprise cutting instructions 328. Preferably, cutting instructions 328 instruct user 602 to remove holder 348 and cut encircler 302 to generate a strip having the circumference of the head of user 602. Preferably, cutting instructions 328 comprise words such as, for example, “Since some printers may be slightly different, we would suggest you cut the hat band on the backside and measure it exactly with a ruler. Then look at the conversion chart for your Hat Band size as well.”, as shown. Upon reading the teachings of this specification, those with ordinary skill in the art will now understand that, under appropriate circumstances, considering issues such as the language of targeted customers, etc., other cutting instruction arrangements may suffice, such as, for example, using different words, providing a diagram, etc.

Preferably, instructions 318 comprise printing instructions 340. Preferably, printing instructions 340 instruct user 602 how to print head encircler kit 300 so that encircler 302 is exactly the correct size. Preferably, printing instructions 340 comprise words such as, for example, “Make sure your printer mode is set at 100% when printing. If you print at less than 100%, your DIGISIZER™ will not be accurate. Example: DIGISIZER™ Toolbox should measure 8 inches long when printed.”. Upon reading the teachings of this specification, those with ordinary skill in the art will now understand that, under appropriate circumstances, considering issues such as the language of targeted customers, data format, etc., other printing instructions (to obtain similar goals) may suffice, such as, for example, using different words, providing other printed size verification means, etc.

FIG. 5 shows a diagram of the encircler 302 portion of head encircler kit 300, prior to assembly. Preferably, head encircler kit 300 comprises cutting indicia 316, as shown, to assist in showing user 602 preferred locations for cutting head encircler kit 300 in order to remove encircler 302 from the remainder of head encircler kit 300. Preferably, cutting indicia 316 comprises lines 317 and scissors symbols 319, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, etc.

Preferably, encircler 302 comprises measure edge 304, as shown. Preferably, measure edge 304 comprises a line, which is located at one end of encircler 302, as shown. Preferably, measure edge 304 is labeled with the words “measure edge” so that users can easily identify it as such. Upon reading the teachings of this specification, those with ordinary skill in the art will now understand that, under appropriate circumstances, considering issues such as language and symbols recognized by targeted customers, etc., other measure edge arrangements may suffice, such as, for example, other types of indicia, using different words to label the measure edge, omitting the words to label the measure edge, using something other than a line for the measure edge (for example, a dot), etc.

Preferably, encircler 302 comprises joint lines 306 and joint edge 366, as shown. Preferably, joint lines 306 and joint edge 366 comprise indicia designed to assist user 602 to assemble encircler 302 with exactly the correct length, as described above in assembly instructions 321. Preferably, joint lines 306 are labeled with the words “joint line” so that users can easily identify them as such. Preferably, joint edge 366 is labeled with the words “joint edge” so that users can easily identify it as such. Upon reading the teachings of this specification, those with ordinary skill in the art will now understand that, under appropriate circumstances, considering issues such as language and symbols recognized by targeted customers, etc., other joint line arrangements may suffice, such as, for example, other types of indicia, using different words to label the joint length, omitting the words to label the joint lines, labeling them joint edge, using something other than a line for the joint lines (for example, a dot), etc.

Preferably, encircler 302 comprises hat size gauge 308. Preferably, hat size gauge 308 marks head size circumferences in centimeters, as measured from the measure edge 304. Preferably, a wide range of circumferences are provided. Most preferably, hat sizes between about 48 centimeters (about hat size 6) and about 68 centimeters (about hat size 8½) are provided. Upon reading the teachings of this specification, those with ordinary skill in the art will now understand that, under appropriate circumstances, considering issues such as the language of targeted customers, etc., other ring size indicia may suffice, such as, for example, using different words, using standard hat sizes, using English measurement, etc.

Preferably, encircler 302 comprises bendable/flexible material so that encircler 302 can be wrapped around the head of user 602, and also so that encircler 302 can be straightened (flattened) out so that it can be conveniently measured by a linear measurement tool, such as, for example, a ruler. Preferably, encircler 302 does not comprise stretchable material, so that changes in length of encircler 302 are negligible in order to insure accurate length measurement. Preferably, encircler 302 is printed onto paper 146, preferably 8½″×11″ paper. Upon reading the teachings of this specification, those with ordinary skill in the art will now understand that, under appropriate circumstances, considering issues such as improvements in print media, and alternatives to paper for printing, etc., other materials may suffice, such as, for example, using film, cloth, synthetic materials, etc.

Preferably, head encircler kit 300 can be printed by a customer by accessing and downloading the data from an Internet website. Preferably, user 602 prints the head encircler kit 300 having head encircler 302 (at least embodying herein wherein such Internet download apparatus comprises at least one encircler) and instructions 318 (at least embodying herein wherein such Internet download apparatus comprises instructions for measuring such wearable article size) on the printer 158 of user 602 (at least embodying herein the step of printing at least one printable head circumference measuring tool onto paper) (as shown in FIG. 7), preferably onto paper 146. Upon reading the teachings of this specification, those of ordinary skill in the art will now understand that, under appropriate circumstances, such as user preference, advances in technology, intended use, etc., various file formats, such as an Adobe Acrobat™ .PDF file, a word processing document, a computerized drawing, an image file, etc., may suffice.

FIG. 6 shows diagrams of several of the instructions described in FIG. 5. In particular, encircler removal instructions 320 (at least embodying herein wherein such first instruction means comprises indicia means for visually illustrating such first instruction means; and at least embodying herein wherein such at least one first instruction comprises at least one indicia adapted to visually illustrate such at least one first instruction), assembly instructions 321, and holder instructions 324 are illustrated.

FIG. 7 provides a conversion chart and an alternate set of measuring instructions. Preferably, instructions 318 comprise conversion chart 350. Preferably, conversion chart 350 provides conversions between the measurement units shown on encircler 302 and another unit of measurement. Preferably, conversion chart 350 converts between centimeters, inches, standard hat sizes, and “character” sizes, as shown. Preferably, instructions 318 comprise conversion chart 351. Preferably, conversion chart 351 provides conversions between centimeters and inches, as shown. Upon reading the teachings of this specification, those with ordinary skill in the art will now understand that, under appropriate circumstances, considering issues such as the language of targeted customers, local units of measurement, etc., other conversion charts (to obtain similar goals) may suffice, such as, for example, using different words, converting ring sizes to English measurements, etc.

Preferably, instructions 318 comprise simplified instructions 360. Preferably, simplified instructions 360 instruct user 602 to measure their hat size with a flexible measuring device already in the possession of user 602, such as, for example, a cloth measuring tape. Preferably, simplified instructions 360 comprise words such as, for example, “Measure around your head about one inch above the eyebrows (considered the widest part of the head) where your hat will be worn. A cloth measuring tape used by tailors/seamstresses is ideal.) It is helpful to have someone assist you. Pull the tape tight to measure within ⅛ of an inch. Write down the measurement number from the tape measure and use the chart below to determine your hat size.”, as shown. Upon reading the teachings of this specification, those with ordinary skill in the art will now understand that, under appropriate circumstances, considering issues such as the language of targeted customers, data format, etc., other simplified instructions (to obtain similar goals) may suffice, such as, for example, using different words, etc.

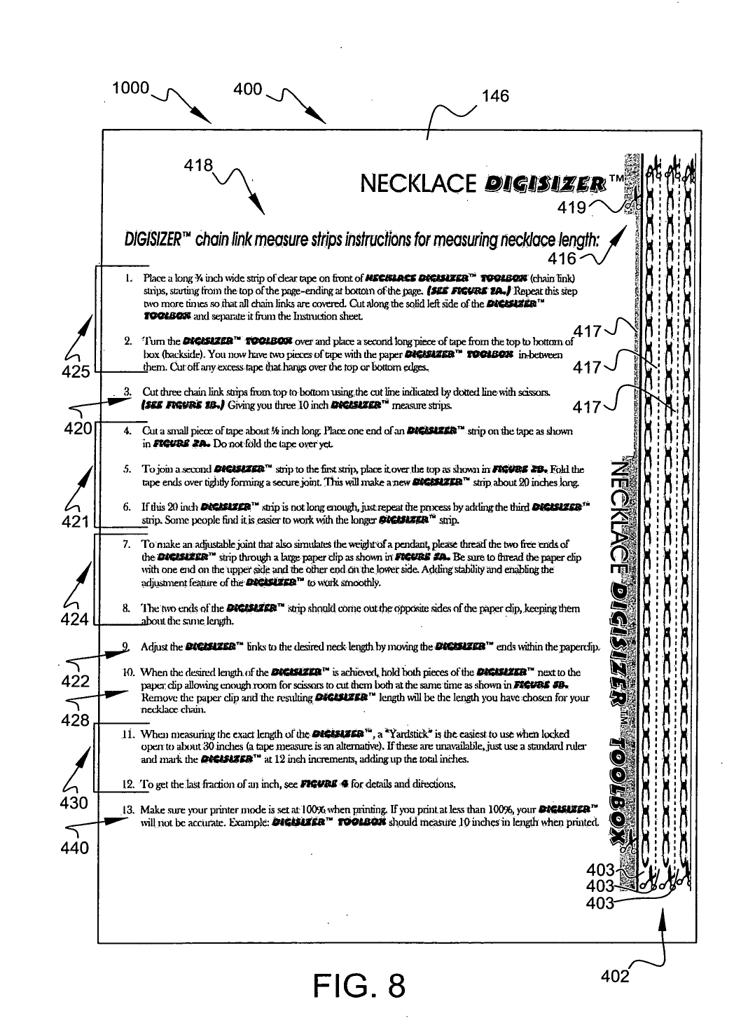

FIG. 8 shows a front view of a portion of a wearable-article sizing system 1000 according to a preferred embodiment of the present invention. Preferably, wearable-article sizing system 1000 comprises at least one neck encircler kit 400.

Preferably, neck encircler kit 400 comprises at least one sheet of paper 146 having printed thereon instructions 418 and at least one encircler 402 (at least embodying herein wherein such encircler means comprises paper; and wherein such at least one encircler comprises at least one paper). Preferably, encircler 402 (at least embodying herein at least one encircler to encircle the neck of the customer) is a long, narrow strip sized to fit at least around the neck of most users. Preferably, encircler 402 (when assembled) is about 30 inches long and about 1/4 inch wide. Preferably, encircler 402 is assembled from three strips 403 that are each about 10 inches long and about 1/4 inch wide, when printed. Upon reading the teachings of this specification, those with ordinary skill in the art will now understand that, under appropriate circumstances, considering issues such as targeted customer, type of necklace or other type of article being sized, etc., other kit and encircler arrangements may suffice, such as, for example, other kit sheet materials than paper, including only a single encircler in an encircling kit, including many encirclers in an encircling kit, different widths and lengths for encirclers, etc.

Preferably, neck encircler kit 400 comprises cutting indicia 416, as shown, to assist in showing user 602 preferred locations for cutting neck encircler kit 400 in order to remove encircler 402 from the remainder of neck encircler kit 400. Preferably, cutting indicia 416 comprises lines 417 and scissors symbols 419, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, etc.

Preferably, instructions 418 comprise lamination instructions 425. Preferably, lamination instructions 425 instruct user 602 to cover both sides (surfaces) of encircler 402 with a laminating material (such as, for example, clear adhesive tape) in order to provide strength and ease of handling to encircler 402 in use. Preferably, lamination instructions 425 comprise words such as, for example, “Place a long 3/4 inch wide strip of clear tape on front of NECKLACE DIGISIZER™ TOOLBox (chain link) strips, starting from the top of the page-ending at bottom of the page. Repeat this step two more times so that all chain links are covered. Cut along the solid left side of the DIGISIZER™ TOOLBox and separate it from the Instruction sheet. Turn the DIGISIZER™ TOOLBox over and place a second long piece of tape from the top to bottom of box (backside). You now have two pieces of tape with the paper DIGISIZER™ TOOLBox in-between them. Cut off any excess tape that hangs over the top or bottom edges.”, as shown. Upon reading the teachings of this specification, those with ordinary skill in the art will now understand that, under appropriate circumstances, considering issues such as the language of targeted customers, etc., other lamination instruction arrangements may suffice, such as, for example, using different words, providing a diagram, etc.

Preferably, instructions 418 comprise encircler removal instructions 420. Preferably, encircler removal instructions 420 instruct user 602 how to remove encircler 402 from neck encircler kit 400. Preferably, encircler removal instructions 420 (at least embodying herein wherein such encircler means is cut out from such printable media means prior to use; and at least embodying herein wherein such at least one encircler is cut out from such at least one printable media prior to use) instruct user 602 to cut neck encircler kit 400. Preferably, encircler removal instructions 420 comprise words such as, for example, “Cut along the solid left side of the Digisizer™ Toolbox and separate it from the Instruction sheet. Cut three chain link strips from top to bottom using the cut line indicated by dotted line with scissors, giving you three 10 inch measure strips.”, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, different words, etc.

Preferably, instructions 418 comprise assembly instructions 421. Preferably, assembly instructions 421 instruct user 602 how to assemble the three strips 403 into one finished encircler 402. Preferably, assembly instructions 421 comprise words such as, for example, “Cut a small piece of tape about ½ inch long. Place one end of a strip on the tape as shown. Do not fold the tape over yet. To join a second strip to the first strip, place it over the top as shown. Fold the tape ends over tightly forming a secure joint. This will make a new strip about 20 inches long. If this 20-inch strip is not long enough, just repeat the process by adding the third strip.”, as shown. Preferably, encircler 402 becomes usable after being cut out of the paper 146 and after being assembled. Upon reading the teachings of this specification, those with ordinary skill in the art will now understand that, under appropriate circumstances, considering issues such as language and symbols recognized by targeted customers, etc., other assembly instructions may suffice, such as, for example, different words, etc.

Preferably, instructions 418 comprise holder instructions 424. Preferably, holder instructions 424 instruct user 602 how to hold encircler 402 in place around the neck of user 602. Preferably, holder instructions 424 (at least embodying herein holder instruction means for instructing the customer in the use of such holder means; and at least embodying herein at least one holder instruction adapted to instruct the customer in the use of such at least one holder) instruct user 602 to hold encircler 402 in place with a holder 448 (at least embodying herein holder means for holding such encircler means in place on the at least one article-wearable body part of the customer; and at least embodying herein at least one holder adapted to hold such at least one encircler in place on the article-wearable body part of the customer) (as shown in FIG. 9), which is preferably a paper clip. Preferably, holder instructions 424 comprise words such as, for example, “To make an adjustable joint that also simulates the weight of a pendant, please thread the two free ends of the strip through a large paper clip as shown. Be sure to thread the paper clip with one end on the upper side and the other end on the lower side. Adding stability and enabling the adjustment feature of the to work smoothly. The two ends of the strip should come out the opposite sides of the paper clip, keeping them about the same length.”, as shown. Upon reading the teachings of this specification, those with ordinary skill in the art will now understand that, under appropriate circumstances, considering issues such as the language of targeted customers, etc., other holder instruction arrangements may suffice, such as, for example, using different words, providing a diagram, etc.

Preferably, instructions 418 comprise encircling instructions 422. Preferably, encircling instructions 422 instruct user 602 how and where to place encircler 402. Preferably, encircling instructions 422 instruct user 602 to encircle their neck with encircler 402 (at least embodying herein wherein such at least one encircler is adapted to encircle the neck of the customer) where user 602 will wear their necklace, at the preferred necklace length. Preferably, encircling instructions 422 comprise words such as, for example, “Adjust the links to the desired neck length by moving the ends within the paperclip.”, as shown. Upon reading the teachings of this specification, those with ordinary skill in the art will now understand that, under appropriate circumstances, considering issues such as the language of targeted customers, etc., other encircling instruction arrangements may suffice, such as, for example, using different words, etc.

Preferably, instructions 418 comprise cutting instructions 428. Preferably, cutting instructions 428 instruct user 602 to remove holder 448 and cut encircler 402 to generate a strip of the circumference of user 602's desired necklace. Preferably, cutting instructions 428 comprise words such as, for example, “When the desired length of the Digisizer™ is achieved, hold both pieces of the Digisizer™ next to the paper clip allowing enough room for scissors to cut them both at the same time, as shown. Remove the paper clip and the resulting length will be the length you have chosen for your necklace chain.”, as shown. These instructions are diagrammed especially in FIG. 9. Upon reading the teachings of this specification, those with ordinary skill in the art will now understand that, under appropriate circumstances, considering issues such as the language of targeted customers, etc., other cutting instruction arrangements may suffice, such as, for example, using different words, etc.

Preferably, instructions 418 comprise measuring instructions 430. Preferably, measuring instructions 430 instruct user 602 how to measure encircler 402 in order to determine the necklace size of user 602. Preferably, measuring instructions 430 instruct user 602 to measure the length of encircler 402 (at least embodying herein wherein such encircler means comprises straightener means for straightening such encircler means; and at least embodying herein wherein such encircler means may be measured by a flat measuring tool; wherein such at least one encircler comprises at least one straightener adapted to straighten such at least one encircler; and at least embodying herein wherein such at least one encircler may be measured by a flat measuring tool) using a linear measuring tool, preferably using a ruler 152, as shown and described herein. Preferably, measuring instructions 430 comprise words such as, for example, “When measuring the exact length of the DIGISIZER™, a “Yardstick” is the easiest to use when locked open to about 30 inches (a tape measure is an alternative). If these are unavailable, just use a standard ruler and mark the DIGISIZER™ at 12 inch increments, adding up the total inches. To get the last fraction of an inch, see “FIG. 4” for details and directions.”, as shown. Upon reading the teachings of this specification, those with ordinary skill in the art will now understand that, under appropriate circumstances, considering issues such as the language of targeted customers, etc., other measuring instruction arrangements (to obtain similar goals) may suffice, such as, for example, using different words, etc.

Preferably, instructions 418 comprise ruler indicia 434. Preferably, ruler indicia 434 show a sample portion of a ruler 152 labeled so that user 602 can easily read and interpret it. Preferably, ruler indicia 434 comprise alignment indicia 435 that shows user 602 where to align the end of encircler 402 on the sample portion of the ruler, as shown. Preferably, alignment indicia 435 comprise words such as, for example, “Start measuring from HERE” and an arrow pointing to the 0″ mark on the sample portion of the ruler, as shown. Upon reading the teachings of this specification, those with ordinary skill in the art will now understand that, under appropriate circumstances, considering issues such as the language of targeted customers, etc., other ruler indicia and alignment indicia arrangements (to obtain similar goals) may suffice, such as, for example, using different words, etc.

Preferably, instructions 418 comprise marker line rules 436. Preferably, marker line rules 436 instruct user 602 how to understand and read the different marker lines on a typical ruler 152. Preferably, marker line rules 436 comprise words such as, for example, “Most rulers follow these ‘marker’ line rules on a one-foot ruler: a) The longest mark line is the inch marker (always located next to the corresponding number indicating inches, 1-12 inches in a foot); b) The 2nd longest mark line is the 1/2 inch marker located exactly halfway between the inch marks; c) The 3rd longest mark line is the 1/4 inch marker located halfway between the 1/2 inch marks; d) The 4th longest mark line is the 1/8 inch marker located halfway between the 1/4 inch marks; 4) The smallest mark line on the ruler is the 1/16 inch marker located halfway between the 1/8 inch marks (we DO NOT size to this small increment).”, as shown. Upon reading the teachings of this specification, those with ordinary skill in the art will now understand that, under appropriate circumstances, considering issues such as the language of targeted customers, etc., other marker line rules arrangements (to obtain similar goals) may suffice, such as, for example, using different words, describing a metric ruler, etc.

Preferably, instructions 418 comprise printing instructions 440. Preferably, printing instructions 440 instruct user 602 how to print neck encircler kit 400 so that encircler 402 is exactly the correct size. Preferably, printing instructions 440 comprise words such as, for example, “Make sure your printer mode is set at 100% when printing. If you print at less than 100%, your DIGISIZER™ will not be accurate. Example: DIGISIZER™ Toolbox should measure 10 inches long when printed.”, as shown. Upon reading the teachings of this specification, those with ordinary skill in the art will now understand that, under appropriate circumstances, considering issues such as the language of targeted customers, data format, etc., other printing instructions (to obtain similar goals) may suffice, such as, for example, using different words, providing another means of verifying the printed size, etc.

FIG. 9 shows diagrams of several of the instructions described in FIG. 8. In particular, encircler removal instructions 420, assembly instructions 421, cutting instructions 428, measuring instructions 430, and holder instructions 424 are illustrated.

Preferably, encircler 402 comprises bendable/flexible material so that encircler 402 can be wrapped around the neck of user 602, and also so that encircler 402 can be straightened (flattened) out so that it can be conveniently measured by a linear measurement tool, such as, for example, a ruler 152. Preferably, encircler 402 does not comprise stretchable material, so that changes in length of encircler 402 are negligible in order to insure accurate length measurement. Preferably, encircler 402 is printed onto paper 146 (at least embodying herein printable media means for printing such encircler means and such first instruction means onto a printable media; and at least embodying herein at least one printable media adapted to printing such at least one encircler and such at least one first instruction onto a printable media), preferably 8½″×11″ paper. Upon reading the teachings of this specification, those with ordinary skill in the art will now understand that, under appropriate circumstances, considering issues such as improvements in print media, and alternatives to paper for printing, etc., other materials may suffice, such as, for example, using film, cloth, synthetic materials, etc.

Preferably, neck encircler kit 400 can be printed by a customer by accessing and downloading the data from an Internet website. Preferably, user 602 prints the neck encircler kit 400 on the printer 158 of user 602 (at least embodying herein the step of printing at least one printable neck circumference measuring tool onto paper; and at least embodying herein wherein such encircler means and such first instruction means are printed onto such printable media means; and at least embodying herein wherein such at least one encircler and such at least one first instruction are printed onto such at least one printable media) (as shown in FIG. 7), preferably onto paper 146 (at least embodying herein wherein such encircler means and such first instruction means comprise downloadable computer-file means for providing a downloadable computer-file from which such encircler means and such instruction means may be printed onto a printable media; and at least embodying herein wherein such at least one encircler and such at least one first instruction comprise at least one downloadable computer-file adapted to provide at least one downloadable computer-file from which such at least one encircler and such at least one instruction may be printed onto a printable media). Upon reading the teachings of this specification, those of ordinary skill in the art will now understand that, under appropriate circumstances, such as user preference, advances in technology, intended use, etc., various file formats, such as an Adobe Acrobat™ .PDF file, a word processing document, a computerized drawing, an image file, etc., may suffice.

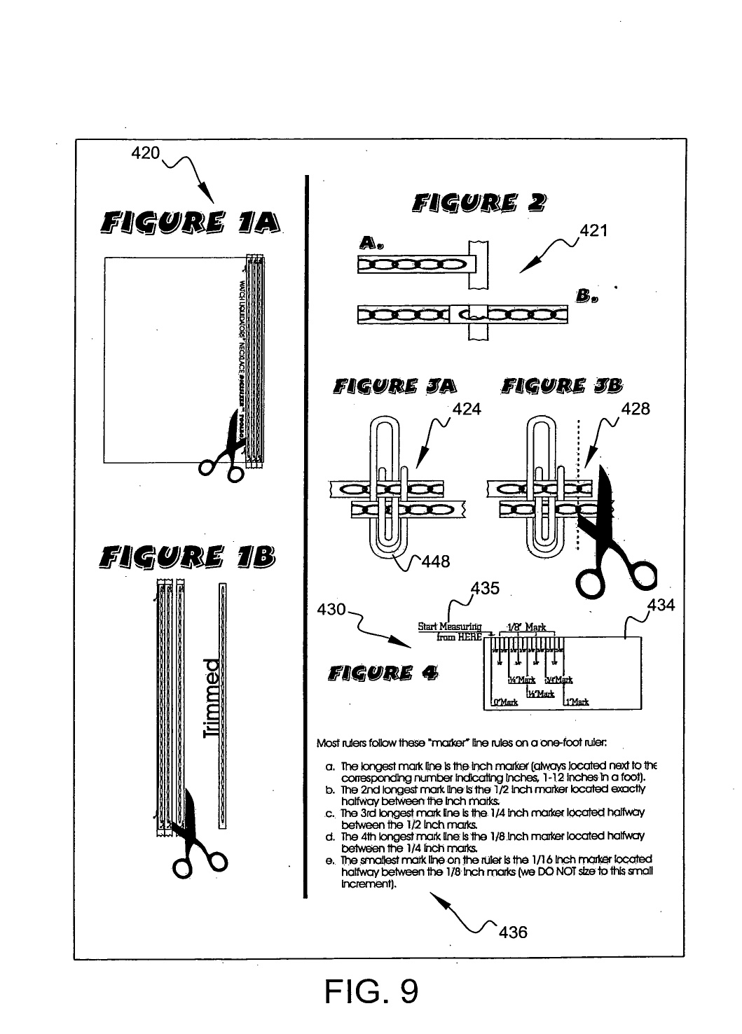

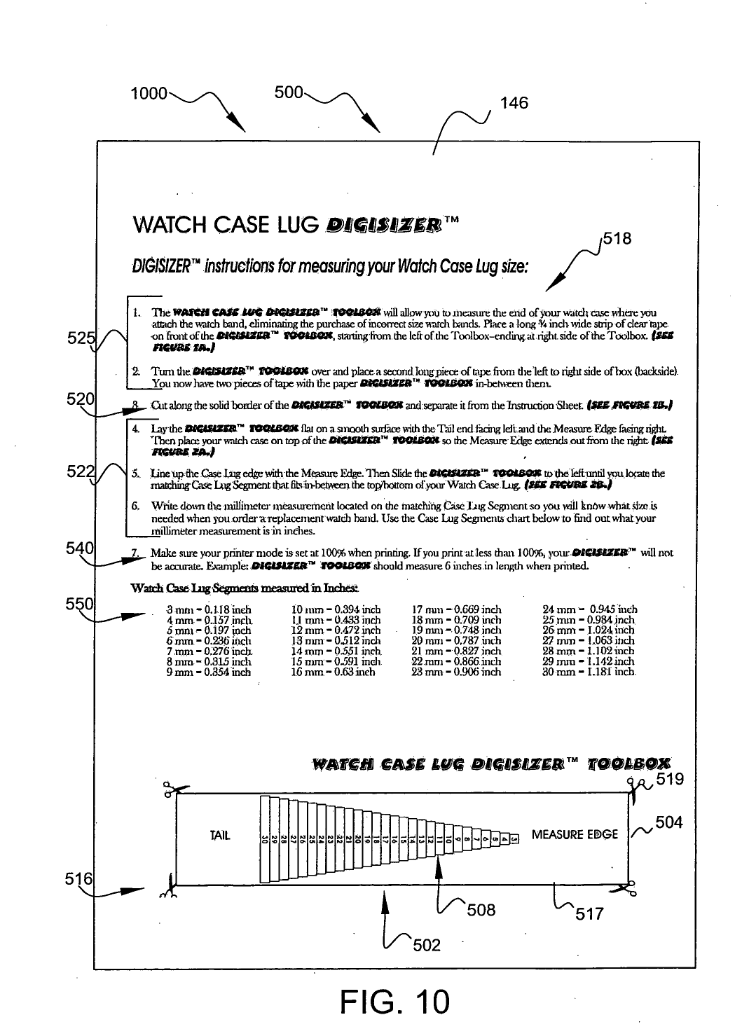

FIG. 10 shows a front view of a portion of a wearable-article sizing system 1000 according to a preferred embodiment of the present invention. Preferably, wearable-article sizing system 1000 comprises at least one watch lug measuring tool kit 500.

Preferably, watch lug measuring tool kit 500 comprises at least one sheet of paper 146 having printed thereon instructions 518 (at least embodying herein at least one first instruction adapted to instruct a customer in the use of such at least one scale) and at least one measuring tool 502. Preferably, measuring tool 502 (at least embodying herein at least one scale to measure a distance between lugs of a watch) is a long, narrow strip having a gauge 508 sized to measure the watch lug of most wristwatches. Preferably, measuring tool 502 is about 1¼ inches wide and about 6 inches long. Upon reading the teachings of this specification, those with ordinary skill in the art will now understand that, under appropriate circumstances, considering issues such as targeted customer, type of article being sized, etc., other kit and measuring tool arrangements may suffice, such as, for example, other kit sheet materials than paper, including only a single measuring tool in a kit, including many measuring tools in a kit, different widths and lengths for measuring tools, etc.

Preferably, watch lug measuring tool kit 500 comprises cutting indicia 516, as shown, to assist in showing user 602 preferred locations for cutting watch lug measuring tool kit 500 in order to remove measuring tool 502 from the remainder of watch lug measuring tool kit 500. Preferably, cutting indicia 516 comprise solid lines 517 and scissors symbols 519, as shown. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, etc.

Preferably, measuring tool 502 comprises measure edge 504, as shown. Preferably, measure edge 504 comprises a line, which is located at one end of measuring tool 502, as shown. Preferably, measure edge 504 is labeled with words such as, for example, “measure edge”, as shown, so that users can easily identify it as such. Upon reading the teachings of this specification, those with ordinary skill in the art will now understand that, under appropriate circumstances, considering issues such as language and symbols recognized by targeted customers, etc., other measure edge arrangements may suffice, such as, for example, other types of indicia, using different words to label the measure edge, omitting the words to label the measure edge, using something other than a line for the measure edge (for example, a dot), etc.

Preferably, measuring tool 502 comprises gauge 508. Preferably, gauge 508 marks standard watch lug widths. Preferably, a wide range of men's and women's watch lug sizes are provided. Most preferably, watch lug sizes between about 3 millimeters and about 30 millimeters are provided. Upon reading the teachings of this specification, those with ordinary skill in the art will now understand that, under appropriate circumstances, considering issues such as the language of targeted customers, etc., other ring size indicia may suffice, such as, for example, using different words, using English measurement, etc.

Preferably, instructions 518 comprise lamination instructions 525. Preferably, lamination instructions 525 instruct user 602 to cover both sides (surfaces) of encircler 502 with a laminating material (such as, for example, clear adhesive tape) in order to provide strength and ease of handling to encircler 502 in use. Preferably, lamination instructions 525 comprise words such as, for example, “Place a long 3/4 inch wide strip of clear tape on front of the DIGISIZER™ TOOLBox, starting from the left of the Toolbox—ending at right side of the Toolbox. Turn the DIGISIZER™ TOOLBox over and place a second long piece of tape from the left to right side of box (backside). You now have two pieces of tape with the paper DIGISIZER™ TOOLBox in-between them.”, as shown. Upon reading the teachings of this specification, those with ordinary skill in the art will now understand that, under appropriate circumstances, considering issues such as the language of targeted customers, etc., other lamination instruction arrangements may suffice, such as, for example, using different words, providing a diagram, etc.

Preferably, instructions 518 comprise measuring tool removal instructions 520. Preferably, measuring tool removal instructions 520 instruct user 602 how to remove measuring tool 502 from watch lug measuring tool kit 500. Preferably, measuring tool removal instructions 520 (at least embodying herein wherein such at least one scale is cut out from such at least one printable media prior to use) instruct user 602 to cut watch lug measuring tool kit 500. Preferably, measuring tool removal instructions 520 comprise words such as, for example, “Cut along the solid border of the DIGISIZER TOOLBox™ and separate it from the Instruction Sheet.”, as shown. Preferably, encircler 502 is usable before and after being cut out of the paper 146. Upon reading the teachings of this specification, those with ordinary skill in the art will now understand that, under appropriate circumstances, considering issues such as language and symbols recognized by targeted customers, etc., other cutting indicia arrangements may suffice, such as, for example, solid lines, different types of dotted and/or dashed lines, omitting scissors symbols, different words, etc.

Preferably, instructions 518 comprise measuring instructions 522. Preferably, measuring instructions 522 instruct user 602 how and where to place a watch lug on measuring tool 502. Preferably, measuring instructions 522 instruct user 602 to measure their watchband width between the watch lugs with measuring tool 502. Preferably, measuring instructions 522 comprise words such as, for example, “Lay the Digisizer™ Toolbox flat on a smooth surface with the Tail end facing left and the Measure Edge facing right. Then place your watchcase on top of the Digisizer™ Toolbox so the Measure Edge extends out from the right. Line up the Case Lug edge with the Measure Edge. Then Slide the Digisizer™ Toolbox to the left until you locate the matching Case Lug Segment that fits in-between the top/bottom of your Watch Case Lug. Write down the millimeter measurement located on the matching Case Lug Segment so you will know what size is needed when you order a replacement watchband.”, as shown. Upon reading the teachings of this specification, those with ordinary skill in the art will now understand that, under appropriate circumstances, considering issues such as the language of targeted customers, etc., other encircling instruction arrangements may suffice, such as, for example, using different words, etc.

Preferably, instructions 518 comprise printing instructions 540. Preferably, printing instructions 540 instruct user 602 how to print watch lug measuring tool kit 500 so that encircler 502 is exactly the correct size. Preferably, printing instructions 540 comprise words such as, for example, “Make sure your printer mode is set at 100% when printing. If you print at less than 100%, your DIGISIZER™ will not be accurate. Example: DIGISIZER™ Toolbox should measure 6 inches in length when printed.”, as shown. Upon reading the teachings of this specification, those with ordinary skill in the art will now understand that, under appropriate circumstances, considering issues such as the language of targeted customers, data format, etc., other printing instructions (to obtain similar goals) may suffice, such as, for example, using different words, providing alternate means of verifying the printed size, etc.

Preferably, instructions 518 comprise conversion chart 550. Preferably, conversion chart 550 provides conversions between the measurement units shown on encircler 502 and another unit of measurement. Preferably, conversion chart 550 provides conversions between millimeters and inches, as shown. Upon reading the teachings of this specification, those with ordinary skill in the art will now understand that, under appropriate circumstances, considering issues such as the language of targeted customers, local units of measurement, etc., other conversion charts (to obtain similar goals) may suffice, such as, for example, using different words, converting to other measurements, etc.

Preferably, measuring tool 502 does not comprise stretchable material, so that changes in the length of measuring tool 502 are negligible in order to insure accurate length measurements. Preferably, measuring tool 502 is printed onto paper 146, preferably 8½″×11″ paper. Upon reading the teachings of this specification, those with ordinary skill in the art will now understand that, under appropriate circumstances, considering issues such as improvements in print media, and alternatives to paper for printing, etc., other materials may suffice, such as, for example, using film, cloth, synthetic materials, etc.

Preferably, watch lug measuring tool kit 500 can be printed by a customer by accessing and downloading the data from an Internet website. Preferably, user 602 prints the watch lug measuring tool kit 500 having measuring tool 502 (at least embodying herein wherein such at least one scale is structured and arranged to be downloaded electronically and printed onto a printable media) and instructions 518 on the printer 158 of user 602 (at least embodying herein the step of printing at least one watch lug width measuring tool onto paper; and at least embodying herein wherein such at least one scale and such at least one first instruction are printed onto such at least one printable media) (as shown in FIG. 7), preferably onto paper 146 (at least embodying herein at least one printable media adapted to printing such at least one scale and such at least one first instruction onto a printable media). Upon reading the teachings of this specification, those of ordinary skill in the art will now understand that, under appropriate circumstances, such as user preference, advances in technology, intended use, etc., various file formats, such as an Adobe Acrobat™ .PDF file, a word processing document, a computerized drawing, an image file, etc., may suffice.

FIG. 11 shows diagrams of several of the instructions described in FIG. 10. In particular, measuring tool removal instructions 520 and measuring instructions 522 are illustrated.

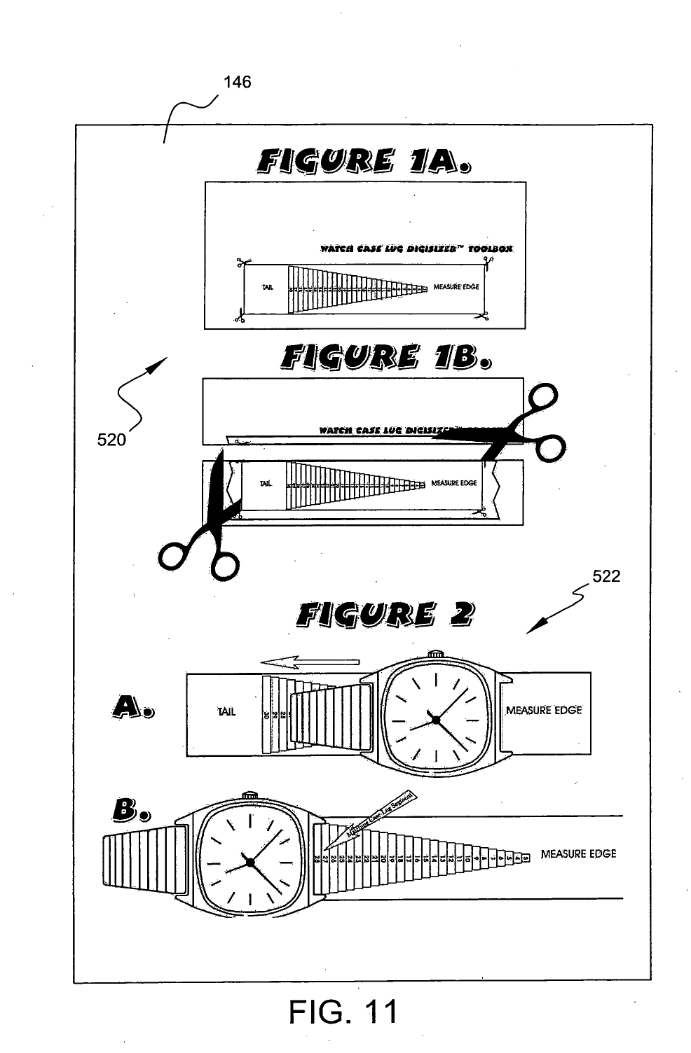

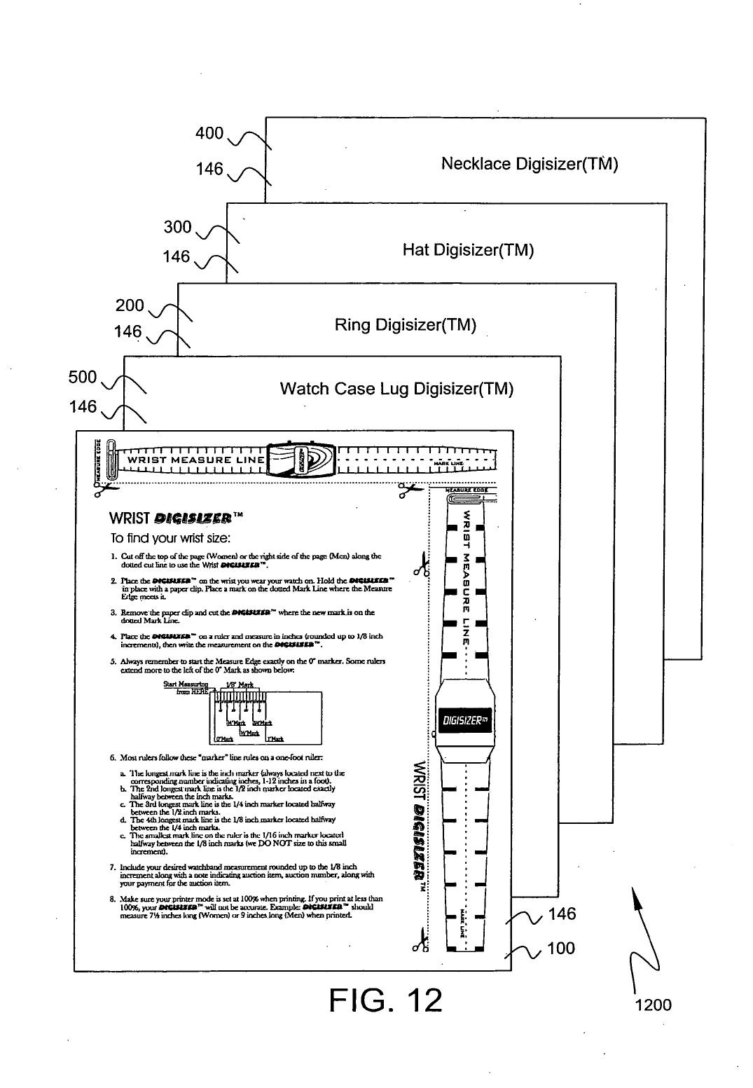

FIG. 12 shows a front view of a wearable-article measuring kit 1200, as printed onto multiple pieces of paper 146. Preferably, wearable-article measuring kit 1200 comprises wrist encircler kit 100 (at least embodying herein at least one downloadable printable wrist circumference measurer), finger encircler kit 200 (at least embodying herein at least one downloadable printable digit circumference measurer), head encircler kit 300 (at least embodying herein at least one downloadable printable head circumference measurer), neck encircler kit 400 (at least embodying herein at least one downloadable printable neck circumference measurer), and watch lug measuring tool kit 500 (at least embodying herein at least one downloadable printable watch lug width measurer). Preferably, wearable-article measuring kit 1200 is downloadable and printable from an Internet location, such as, for example, a website or an Internet directory. Preferably, wearable-article measuring kit 1200 comprises a single downloadable file. More preferably, wearable-article measuring kit 1200 comprises multiple downloadable files accessible at the same Internet or file location. Upon reading the teachings of this specification, those of ordinary skill in the art will now understand that, under appropriate circumstances, such as user preference, advances in technology, intended use, type of vendor, etc., other wearable-article measuring kits, such as kits having more measuring devices, kits having fewer measuring devices, etc., may suffice.

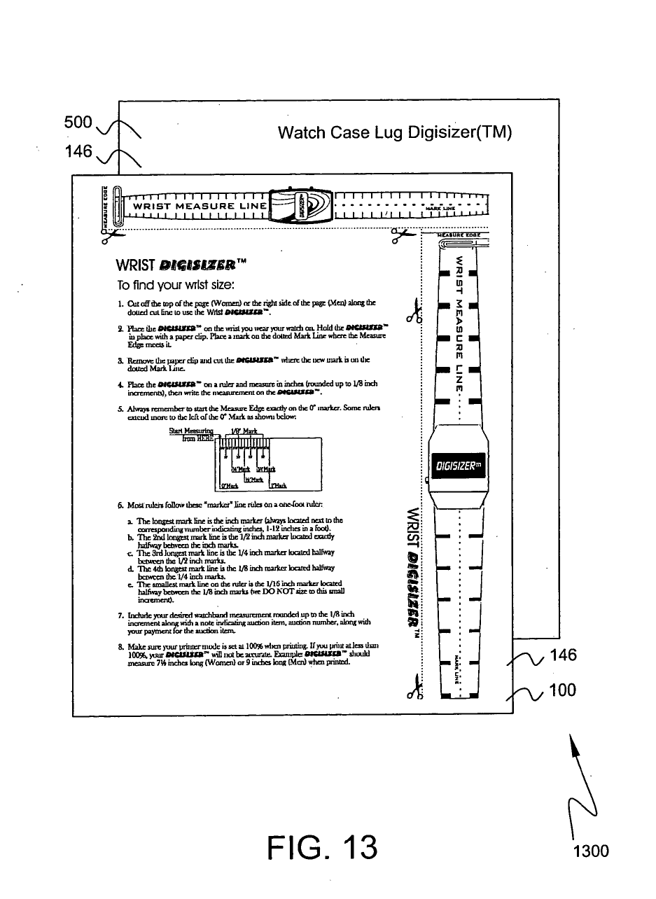

FIG. 13 shows a front view of a watch-measuring kit 1300, as printed onto paper 146. Preferably, watch measuring kit 1300 comprises wrist encircler kit 100 and watch lug measuring tool kit 500. Preferably, watch-measuring kit 1300 is downloadable and printable from an Internet location, such as, for example, a website or an Internet directory. Preferably, watch-measuring kit 1300 comprises a single downloadable file. More preferably, watch-measuring kit 1300 comprises multiple downloadable files accessible at the same Internet location. Upon reading the teachings of this specification, those of ordinary skill in the art will now understand that, under appropriate circumstances, such as user preference, advances in technology, intended use, type of vendor, etc., other wearable-article measuring kits, such as kits having more measuring devices, kits having fewer measuring devices, etc., may suffice.

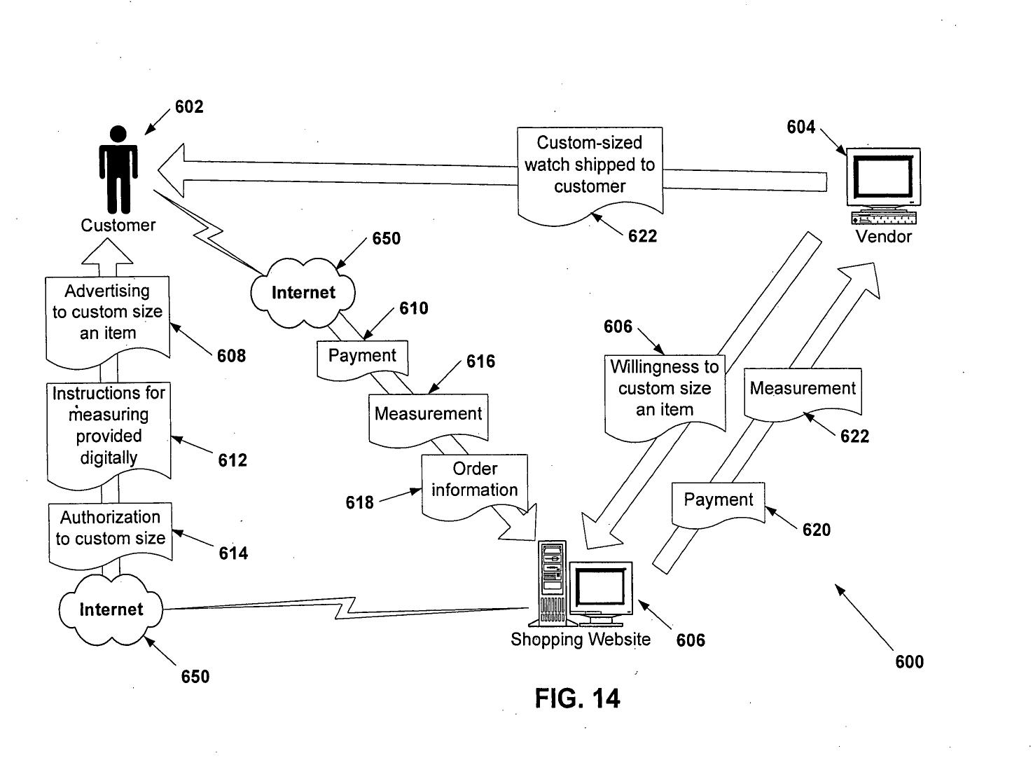

FIG. 14 is a simplified relational diagram of preferred steps in a method 600 of using a wearable-article sizing system 1000 according to a preferred embodiment of the present invention. Methods 600, 700, and 800 are described here using wrist encircler kit 100 as an example, but methods 600, 700, and 800 are equally applicable to any component of wearable-article sizing system 1000, such as, for example, finger encircler kit 200, head encircler kit 300, neck encircler kit 400, and watch lug measuring tool kit 500, wearable-article measuring kit 1200, and watch measuring kit 1300.

Often customers 602 buy watches online from a shopping website 606 (such as, for example, an Internet auction site like ebay®, swatch®, etc.) Preferably, vendor 604 provides watches to be sold by shopping website 606 on Internet 650.

Preferably, vendor 604 informs shopping website 606 whether or not custom sizing is available for a particular watch (or other wearable article, etc.) to be sold, as indicated by flow 606. For example, it may not be possible to size some watches, or some vendors may not have the capability to size some watches, etc. Preferably, custom sizing is available and provided by vendor 604. Preferably, (if custom sizing is available) shopping website 606 advertises and/or notifies customer 602 that custom sizing is available for the watch that the customer has purchased (or is interested in purchasing), as indicated by flow 608 (at least embodying herein such vendor advertising the availability of custom sizing). For example, if shopping website 606 (for example, ebay®) is auctioning or selling a watch, shopping website 606 preferably advertises (such as, for example, in the description of the watch being auctioned) that optional custom sizing is available for an additional fee added to the winning bid price or base sale price, as indicated by flow 608. Preferably, shopping website 606 also notifies/reminds customer 602 (winner of the auction, etc.) that optional custom sizing is available and presents customer 602 with the option for custom sizing. Upon reading the teachings of this specification, those with ordinary skill in the art will now understand that, under appropriate circumstances, considering issues such as type of shopping website, customer preference, vendor preference, etc., other custom sizing advertising and notification arrangements may suffice, such as, for example, sending the customer a separate e-mail with the custom sizing offer, etc.

Preferably, customer 602 accepts optional custom sizing by clicking a button on shopping website 606. Preferably, customer 602 pays the fee for custom sizing to shopping website 606, as indicated by flow 610 (at least embodying herein the step of (the customer) paying the vendor a fee to adjust the size of the wearable article; and at least embodying herein such vendor receiving a fee from a customer for custom sizing). Upon reading the teachings of this specification, those with ordinary skill in the art will now understand that, under appropriate circumstances, considering issues such as type of shopping website, customer preference, vendor preference, etc., other payment arrangements may suffice, such as, for example, bundling the price of custom sizing in with the price of the watch, etc.

Preferably, after customer 602 has elected and paid for custom sizing, customer 602 is granted authorization to upload their measurement information for custom sizing as indicated by flow 614 (at least embodying herein electronically providing measuring instructions to the customer for a method of measuring the article-wearable body part of the customer; and at least embodying herein such vendor providing the customer with electronically downloadable and printable instructions for measuring such customer's proper size), and customer 602 is given access to electronically download instructions for measuring (preferably, by downloading wrist encircler kit 100), as indicated by step 612 (at least embodying herein the step of (the customer) downloading such measuring instructions from an Internet website; and at least embodying herein providing to such customer having a particular wearable-article size an Internet download apparatus for measuring such wearable-article size). Upon reading the teachings of this specification, those with ordinary skill in the art will now understand that, under appropriate circumstances, considering issues such as type of shopping website, customer preference, vendor preference, etc., other access arrangements may suffice, such as, for example, allowing the customer to access encircler kit before paying, etc.

Preferably, customer 602 follows the instructions for measuring and provides the resulting measurement to shopping website 606 as indicated by flow 616 (at least embodying herein having the customer provide the measurement to the vendor; and at least embodying herein providing an upload opportunity for such customer to provide to such vendor a particular wearable-article size for such purchased wearable article; and at least embodying herein such vendor receiving such customer's proper size information). Preferably, customer 602 provides order and/or authorization information identifying the watch associated with the measurement (and that custom sizing has been authorized), as indicated by flow 618. Preferably, shopping website 606 retains a portion of payment 610 (as commission and/or incentive for facilitating the service) and forwards the balance of payment 610 to vendor 604, as indicated by flow 620. Preferably, shopping website 606 also forwards the measurement and authorization information to vendor 604, as indicated by flow 622. According to an alternate preferred embodiment, vendor 604 owns and/or operates shopping website 606, further streamlining the process of forwarding payments and measurement, etc., to vendor 604. Preferably, vendor 604 custom sizes the wearable article based on the measurement provided by customer 602 and ships the custom sized wearable article to the customer 602, as indicated by flow 622 (at least embodying herein sizing at least one wearable article based on such measurement; and at least embodying herein shipping such at least one wearable article to the customer; and at least embodying herein wherein the step of sizing at least one wearable article comprises the step of sizing at least one bracelet; and at least embodying herein the step of such vendor adjusting at least one bracelet to such customer's proper size; and at least embodying herein wherein the step of sizing at least one wearable article comprises the step of sizing at least one hat; and at least embodying herein the step of such vendor adjusting at least one head-wearable article to such customer's proper size; and at least embodying herein wherein the step of sizing at least one wearable article comprises the step of sizing at least one ring; and at least embodying herein the step of such vendor adjusting at least one ring to such customer's proper size; and at least embodying herein wherein the step of sizing at least one wearable article comprises the step of sizing at least one necklace; and at least embodying herein the step of such vendor adjusting at least one neck-wearable article to such customer's proper size). Upon reading the teachings of this specification, those of ordinary skill in the art will now understand that, under appropriate circumstances, such as user preference, advances in technology, intended use, etc., other steps, such as vendors providing unrestricted customer access to the system, etc., may suffice.

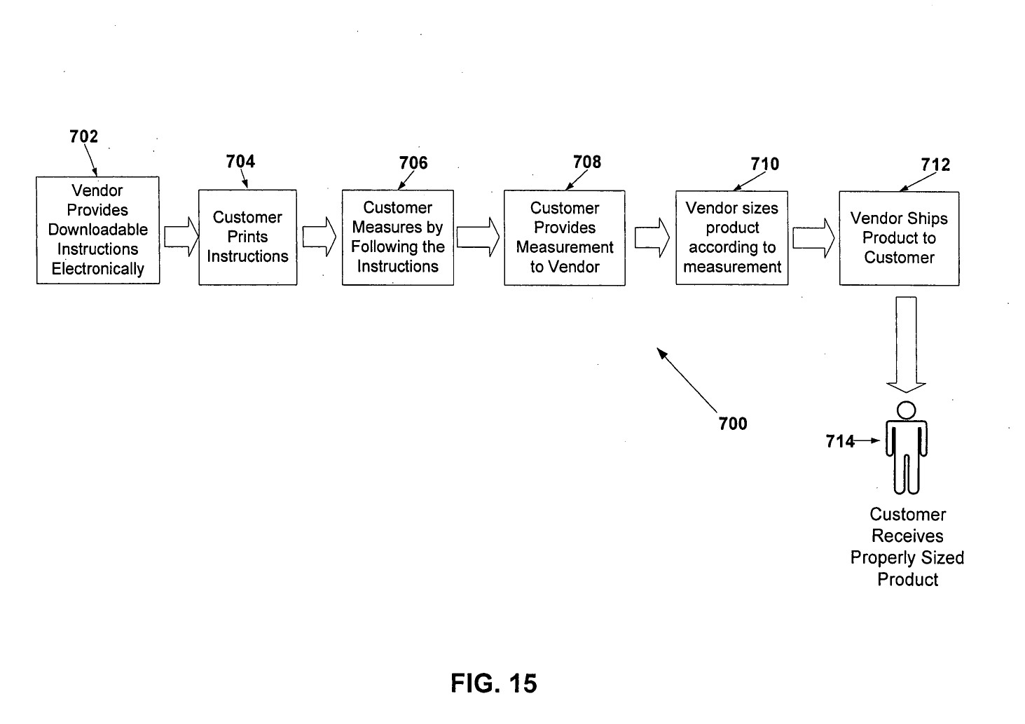

FIG. 15 is a simplified flow diagram of preferred steps in a method 700 of utilizing a wearable-article sizing system 1000 according to a preferred embodiment of the present invention. Preferably, vendor 604 provides instructions to customer 602 (instructing the customer 602 how to measure their wrist) as indicated by step 702. Preferably, the customer 602 prints the instructions as indicated by step 704 (as shown in FIG. 7). Preferably, customer 602 measures wrist size by following the instructions provided as indicated by step 706. Preferably, customer 602 provides the measurement to vendor 604, as indicated by step 708. Preferably, vendor 604 custom sizes the wristband to the size provided by customer 602 as indicated by step 710 (at least embodying herein wherein the step of sizing at least one wearable article comprises the step of sizing at least one wristband; and at least embodying herein the step of such vendor adjusting at least one wrist-wearable article to such customer's proper size; and at least embodying herein such vendor adjusting such at least one wearable article to such customer's proper size). Preferably, vendor 604 ships the product to customer 602 as indicated by step 712 (at least embodying herein such vendor shipping such at least one wearable article to such customer). And, finally, the customer 602 receives the properly sized wrist-wearable article as indicated by step 714. Upon reading the teachings of this specification, those of ordinary skill in the art will now understand that, under appropriate circumstances, such as user preference, advances in technology, intended use, etc., other steps, such as vendors providing unrestricted customer access to the system, etc., may suffice.

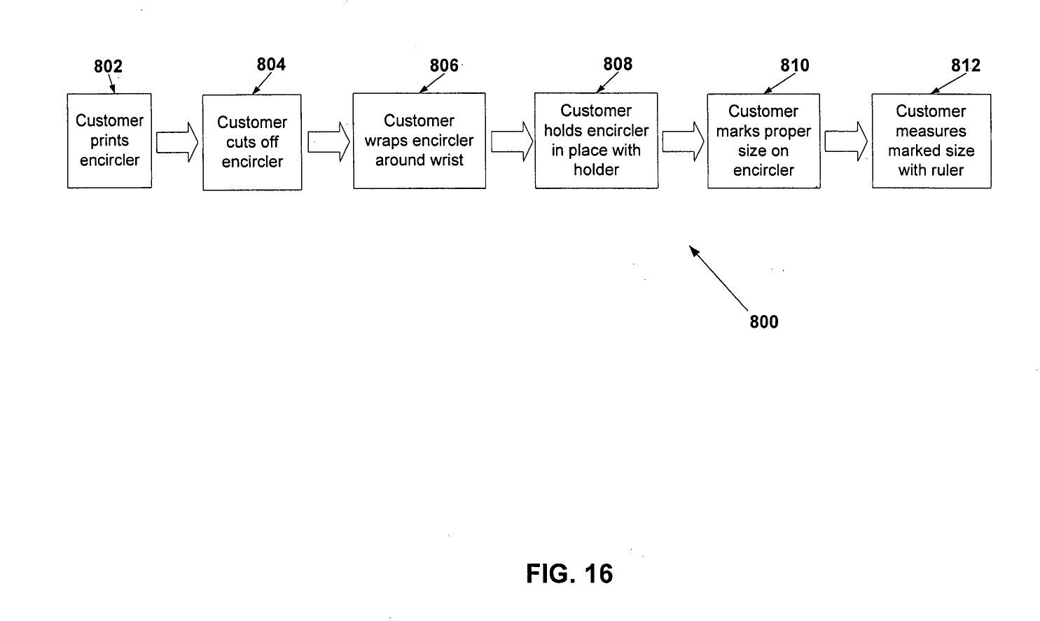

FIG. 16 is a simplified flow diagram of preferred steps in a method 800 of utilizing a wearable-article sizing system 1000 according to a preferred embodiment of the present invention. Preferably, customer 602 prints encircler 102 (preferably, by printing wrist encircler kit 100) as indicated by step 802 (at least embodying herein the step of (the customer) printing an encircler on a printer). Preferably, customer 602 cuts (preferably, with scissors 184) encircler 102 from excess paper 146 as indicated by step 804. Preferably, customer 602 wraps encircler 102 around wrist as indicated by step 806. Preferably, customer 602 holds encircler 102 in place with holder 148, which is preferably a paper clip, as indicated by step 808. Preferably, customer 602 makes mark 150 (preferably, with pen 186) on encircler 102 at the location of the proper size as indicated by step 810. Preferably, customer 602 cuts encircler 102 at the marked location. Preferably, customer 602 removes encircler 102 from the wrist, places encircler 102 on a flat surface, and measures the cut (or marked) size on encircler 102 with a ruler 182, as indicated by step 812. Upon reading the teachings of this specification, those of ordinary skill in the art will now understand that, under appropriate circumstances, such as user preference, advances in technology, intended use, etc., other steps, such as vendors providing unrestricted customer access to the system, etc., may suffice.

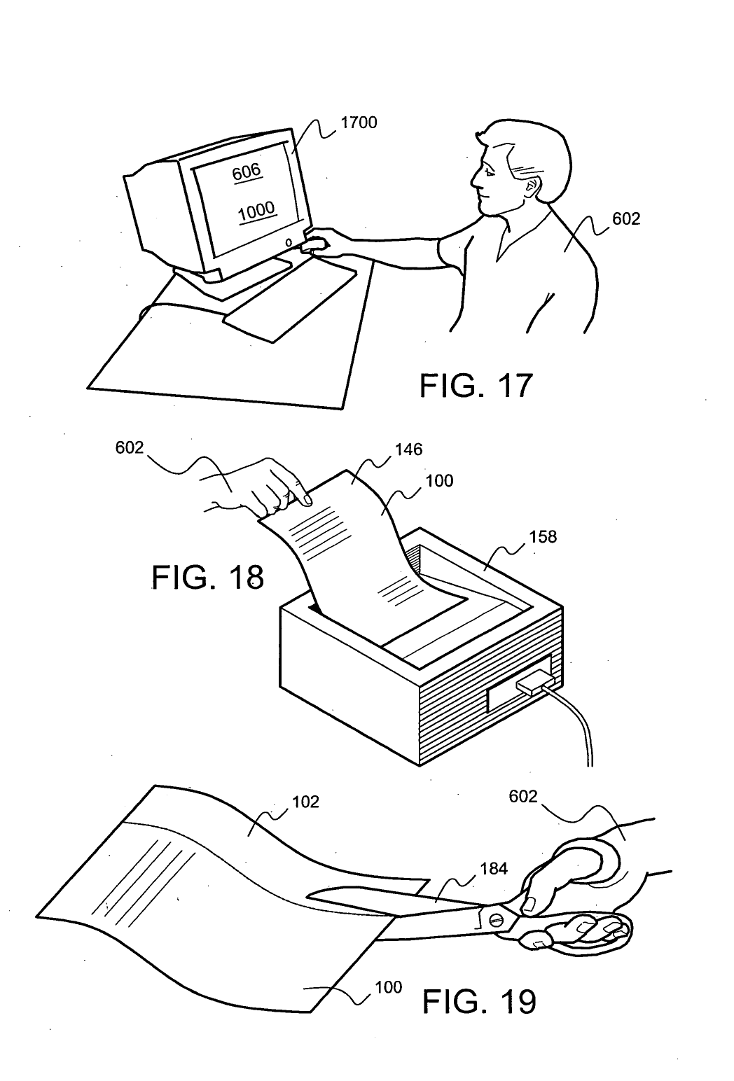

FIG. 17 is a perspective view of customer 602 downloading a wearable-article sizing system 1000 from shopping website 606 using a computer 1700 (at least embodying herein such customer electronically downloading and printing instructions for measuring such customer's proper size).

FIG. 18 is a perspective view of customer 602 using printer 158 to print wrist encircler kit 100 (with encircler 102) onto paper 146 (at least embodying herein the step of (the customer) printing such measuring instruction on a printer; and printing such at least one printable measuring tool onto paper).

FIG. 19 is a perspective view of customer 602 cutting encircler 102 from wrist encircler kit 100 using scissors 184 (at least embodying herein cutting such at least one printable measuring tool out of the paper).

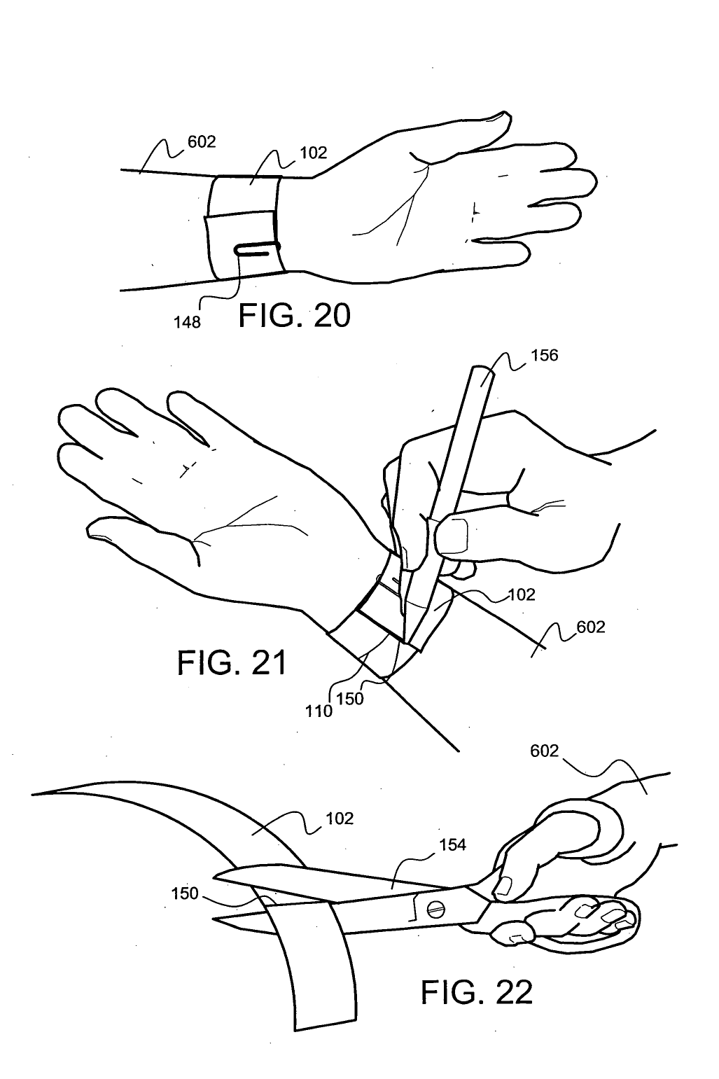

FIG. 20 is a perspective view of encircler 102 (at least embodying herein wrapping at least one flexible object around the article-wearable body part of the customer), which has been encircled around the wrist of customer 602 and held in place with holder 148 (at least embodying herein wherein such at least one holder comprises at least one paper clip) (in this example, a paper clip).

FIG. 21 is a perspective view of customer 602 making mark 150 of their wrist size on mark line 110 of encircler 102 using pen 156 (at least embodying herein marking such at least one flexible object).

FIG. 22 is a perspective view of customer 602 cutting encircler 102 at mark 150 using scissors 154 (at least embodying herein removing such at least one flexible object from the article-wearable body part of the customer).

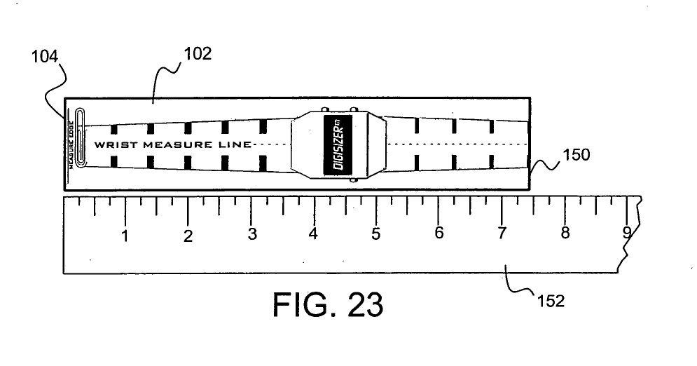

FIG. 23 is a perspective view of encircler 102 being measured from measure edge 104 to mark 150 using ruler 152 (at least embodying herein laying such at least one flexible object out flat, measuring the marked length of such at least one flexible object).

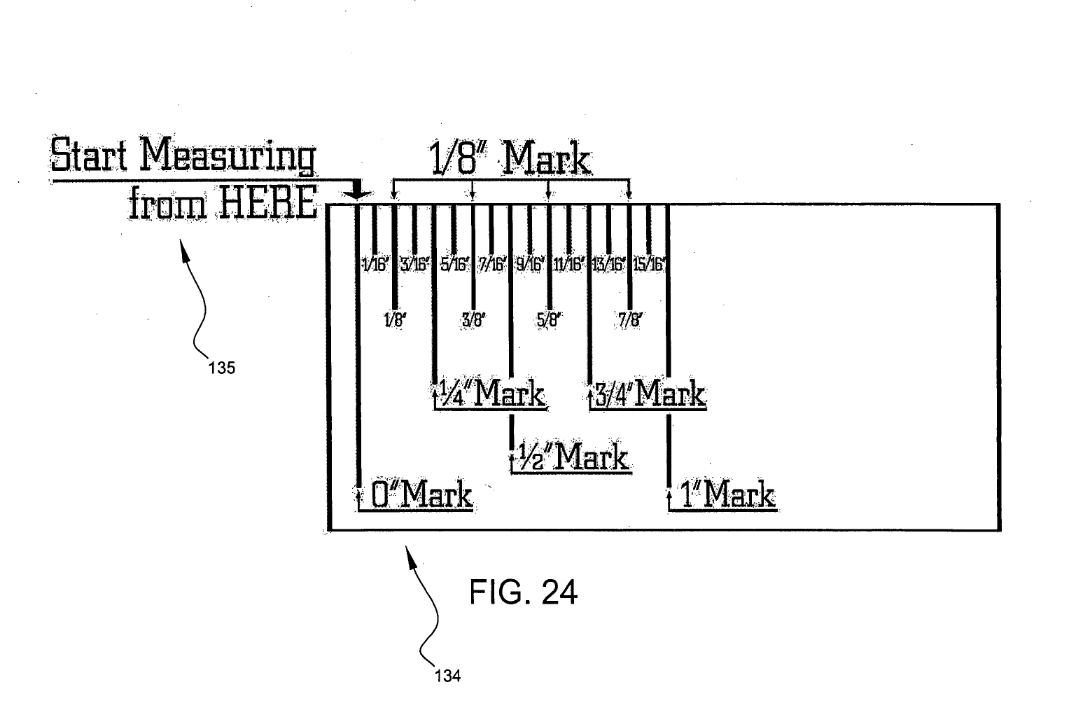

FIG. 24 is an enlarged view of the ruler indicia 134 and alignment indicia 135 according to FIG. 1.

Although applicant has described applicant's preferred embodiments of this invention, it will be understood that the broadest scope of this invention includes such modifications as diverse shapes and sizes and materials. Such scope is limited only by the below claims as read in connection with the above specification.

Further, many other advantages of applicant's invention will be apparent to those skilled in the art from the above descriptions and the below claims.

1) A size measuring system, for allowing at least one customer to receive at least one properly sized wearable article shipped from at least one vendor, comprising:
a) encircler means for encircling at least one article-wearable body part of the at least one customer; and
b) first instruction means for instructing the at least one customer in the use of said encircler means to provide a measurement for the wearable article;
c) wherein said encircler means comprises straightener means for straightening said encircler means, wherein said encircler means may be measured by a flat measuring tool; and
d) wherein said encircler means and said first instruction means comprise downloadable computer-file means for providing a downloadable computer-file from which said encircler means and said instruction means may be printed onto a printable media.

2) The size measuring system, according to claim 1, further comprising:
a) printable media means for printing said encircler means and said first instruction means onto a printable media;
b) wherein said encircler means and said first instruction means are printed onto said printable media means; and
c) wherein said encircler means is cut out from said printable media means prior to use.

3) The size measuring system, according to claim 1, further comprising:
a) bolder means for holding said encircler means in place on the at least one article-wearable body part of the customer; and
b) holder instruction means for instructing the customer in the use of said holder means.

4) The size measuring system, according to claim 1, wherein said encircler means comprises paper.

5) The size measuring system, according to claim 1, wherein said first instruction means comprises indicia means for visually illustrating said first instruction means.

6) The size measuring system, according to claim 1, wherein said first instruction means comprises ruler instruction means for instructing the customer in the use of a ruler to measure a length of said encircler means.

7) The size measuring system, according to claim 6, wherein said ruler instruction means comprises ruler indicia means for visually illustrating the use of a ruler to measure a length of said encircler means.

8) A size measuring system, for allowing at least one customer to receive at least one properly sized wearable article shipped from at least one vendor, comprising:
a) at least one encircler adapted to encircle an article-wearable body part of the customer; and
b) at least one first instruction adapted to instruct a customer in the use of said at least one encircler to provide a measurement for the wearable article;
c) wherein said at least one encircler comprises at least one straightener adapted to straighten said at least one encircler, wherein said at least one encircler may be measured by a flat measuring tool; and
d) wherein said at least one encircler and said at least one first instruction comprise at least one downloadable computer-file adapted to provide at least one downloadable computer-file from which said at least one encircler and said at least one instruction may be printed onto a printable media.

9) The size measuring system, according to claim 8, further comprising:
a) at least one printable media adapted to printing said at least one encircler and said at least one first instruction onto a printable media;
b) wherein said at least one encircler and said at least one first instruction are printed onto said at least one printable media; and
c) wherein said at least one encircler is cut out from said at least one printable media prior to use.

10) The size measuring system, according to claim 8, further comprising
a) at least one holder adapted to hold said at least one encircler in place on the article-wearable body part of the customer; and
b) at least one holder instruction adapted to instruct the customer in the use of said at least one holder.

11) The size measuring system, according to claim 10, wherein said at least one holder comprises at least one paper clip.

12) The size measuring system, according to claim 10, wherein said at least one holder comprises at least one piece of adhesive tape.

13) The size measuring system, according to claim 8, wherein said at least one encircler comprises at least one paper.

14) The size measuring system, according to claim 8, wherein said at least one first instruction comprises at least one indicia adapted to visually illustrate said at least one first instruction.

15) The size measuring system, according to claim 8, wherein said at least one instructions comprises
a) at least one ruler instructions adapted to instruct the customer in the use of a ruler to measure a length of said at least one encircler; and
b) at least one ruler indicia adapted to visually illustrate said at least one ruler instructions.

16) The size measuring system, according to claim 8, wherein said at least one encircler is adapted to encircle at least one wrist of the customer.

17) The size measuring system, according to claim 8, wherein said at least one encircler is adapted to encircle the head of the customer.

18) The size measuring system, according to claim 8, wherein said at least one encircler is adapted to encircle at least one digit of the customer.

19) The size measuring system, according to claim 8, wherein said at least one encircler is adapted to encircle the neck of the customer.

20) A size measuring system, for allowing a customer to receive a properly sized wrist-wearable article shipped from a vendor, comprising:
a) at least one encircler to encircle a wrist of the customer;
b) wherein said at least one encircler is adapted to be straightened, wherein said at least one encircler may be measured by a linear measuring tool; and
c) wherein said at least one encircler is structured and arranged to be downloaded electronically and printed.

21) The size measuring system, according to claim 20, further comprising at least one first instructions adapted to instruct the customer in the use of said at least one encircler.

22) The size measuring system, according to claim 20, further comprising at least one indicium adapted to visually assist the customer to use said at least one encircler.

23) A size measuring system, for allowing a customer to receive a properly sized head-wearable article shipped from a vendor, comprising:
a) at least one encircler to encircle the head of the customer;
b) wherein said at least one encircler is adapted to be straightened, wherein said at least one encircler may be measured by a linear measuring tool; and
c) wherein said at least one encircler is structured and arranged to be downloaded electronically and printed.

24) The size measuring system, according to claim 23, further comprising at least one first instructions adapted to instruct the customer in the use of said at least one encircler.

25) The size measuring system, according to claim 23, further comprising at least one indicium adapted to visually assist the customer to use said at least one encircler.

26) A size measuring system, for allowing a customer to receive a properly sized ring shipped from a vendor, comprising:
a) at least one encircler to encircle a digit of the customer,
b) wherein said at least one encircler is adapted to be straightened, wherein said at least one encircler may be measured by a linear measuring tool; and
c) wherein said at least one encircler is structured and arranged to be downloaded electronically and printed.

27) The size measuring system, according to claim 26, further comprising at least one first instructions adapted to instruct the customer in the use of said at least one encircler.

28) The size measuring system, according to claim 26, further comprising at least one indicium adapted to visually assist the customer to use said at least one encircler.

29) A size measuring system, for allowing a customer to receive a properly sized neck-wearable article shipped from a vendor, comprising:
a) at least one encircler to encircle the neck of the customer;
b) wherein said at least one encircler is adapted to be straightened, wherein said at least one encircler may be measured by a linear measuring tool; and
c) wherein said at least one encircler is structured and arranged to be downloaded electronically and printed.

30) The size measuring system, according to claim 29, further comprising at least one first instructions adapted to instruct the customer in the use of said at least one encircler.

31) The size measuring system, according to claim 29, further comprising at least one indicium adapted to visually assist the customer to use said at least one encircler.

32) A method, for allowing a customer to receive a properly sized wearable article shipped from a vendor, comprising the steps of:
a) electronically providing measuring instructions to the customer for a method of measuring the article-wearable body part of the customer;
b) wherein such instructions comprise the steps of:
i) wrapping at least one flexible object around the article-wearable body part of the customer,
ii) marking such at least one flexible object,
iii) removing such at least one flexible object from the article-wearable body part of the customer,
iv) laying such at least one flexible object out flat,
v) measuring the marked length of such at least one flexible object;
c) having the customer provide the measurement to the vendor;
d) sizing at least one wearable article based on such measurement; and
e) shipping such at least one wearable article to the customer.

33) The method, according to claim 32, further comprising the step of (the customer) downloading such measuring instructions from an Internet website.

34) The method, according to claim 32, further comprising the step of (the customer) printing such measuring instruction on a printer.

35) The method, according to claim 32, further comprising the step of (the customer) printing an encircler on a printer.

36) The method, according to claim 32, further comprising the step of (the customer) paying the vendor a fee to adjust the size of the wearable article.

37) The method, according to claim 32, wherein the step of sizing at least one wearable article comprises the step of sizing at least one wristband.

38) The method, according to claim 32, wherein the step of sizing at least one wearable article comprises the step of sizing at least one bracelet.

39) The method, according to claim 32, wherein the step of sizing at least one wearable article comprises the step of sizing at least one hat.

40) The method, according to claim 32, wherein the step of sizing at least one wearable article comprises the step of sizing at least one ring.

41) The method, according to claim 31, wherein the step of sizing at least one wearable article comprises the step of sizing at least one necklace.

42) A method, relating to providing properly sized wearable articles by vendors to customers, comprising the steps of:
a) providing to such customer having a particular wearable-article size an electronic download apparatus for measuring such wearable-article size; and
b) providing an upload opportunity for such customer to provide to such vendor a particular wearable-article size for such purchased wearable article;
c) wherein such electronic download apparatus comprises at least one encircler
d) wherein such electronic download apparatus comprises instructions for measuring such wearable-article size.

43) (cancelled).

44) (canceled).

45) A method, for allowing at least one customer to receive at least one properly sized wearable article shipped from at least one vendor, comprising the steps of:
a) such vendor advertising the availability of custom sizing;
b) such vendor receiving a fee from a customer for custom sizing;
c) such vendor providing the customer with electronically downloadable and printable instructions for measuring such customer's proper size;
i) wherein such instructions comprise at least one encircler;
d) such vendor receiving such customer's proper size information;
e) such vendor adjusting such at least one wearable article to such customer's proper size; and
f) such vendor shipping such at least one wearable article to such customer.

46) The method, according to claim 45, wherein the step of such vendor adjusting such at least one wearable article to such customer's proper size comprises the step of such vendor adjusting at least one wrist-wearable article to such customer's proper size.

47) The method, according to claim 45, wherein the step of such vendor adjusting such at least one wearable article to such customer's proper size comprises the step of such vendor adjusting at least one bracelet to such customer's proper size.

48) The method, according to claim 45, wherein the step of such vendor adjusting such at least one wearable article to such customer's proper size comprises the step of such vendor adjusting at least one head-wearable article to such customer's proper size.

49) The method, according to claim 45, wherein the step of such vendor adjusting such at least one wearable article to such customer's proper size comprises the step of such vendor adjusting at least one ring to such customer's proper size.

50) The method, according to claim 45, wherein the step of such vendor adjusting such at least one wearable article to such customer's proper size comprises the step of such vendor adjusting at least one neck-wearable article to such customer's proper size.

51) A size measuring system, for allowing a customer to receive a properly sized watchband shipped from a vendor, comprising:
a) at least one scale to measure a distance between lugs of a watch;
b) at least one first instruction adapted to instruct a customer in the use of said at least one scale;
c) wherein said at least one scale is structured and arranged to be downloaded electronically and printed onto a printable media;
d) at least one printable media adapted to printing said at least one scale and said at least one first instruction onto a printable media;
e) wherein said at least one scale and said at least one first instruction are printed onto said at least one printable media; and
f) wherein said at least one scale is cut out from said at least one printable media prior to use.

52) (cancelled)

53) A method, for allowing at least one customer to make at least one durable measuring device, comprising the steps of:
a) such customer electronically downloading and printing instructions for measuring such customer's proper size;
i) wherein such instructions comprise at least one printable measuring tool;
ii) wherein such instructions comprise the step of applying transparent tape to the front and back surfaces of such at least one printable measuring tool;
b) printing such at least one printable measuring tool onto paper;
c) applying transparent tape to the front and back surfaces of such at least one printable measuring tool; and
d) cutting such at least one printable measuring tool out of the paper.

54) The method, according to claim 53, wherein the step of printing such at least one printable measuring tool onto paper comprises the step of printing at least one printable wrist circumference measuring tool onto paper.

55) The method, according to claim 53, wherein the step of printing such at least one printable measuring tool onto paper comprises the step of printing at least one printable head circumference measuring tool onto paper.

56) The method, according to claim 53, wherein the step of printing such at least one printable measuring tool onto paper comprises the step of printing at least one printable digit circumference measuring tool onto paper.

57) The method, according to claim 53, wherein the step of printing such at least one printable measuring tool onto paper comprises the step of printing at least one printable neck circumference measuring tool onto paper.

58) The method, according to claim 53, wherein the step of printing such at least one printable measuring tool onto paper comprises the step of printing at least one watch lug width measuring tool onto paper.

59) A kit, for allowing a customer to receive a properly sized article shipped from a vendor, comprising:
a) at least one downloadable printable wrist circumference measurer,
b) at least one downloadable printable head circumference measurer;
c) at least one downloadable printable digit circumference measurer;
d) at least one downloadable printable neck circumference measurer; and
e) at least one downloadable printable watch lug width measurer.

60) A kit, for allowing a customer to receive a properly sized article shipped from a vendor, comprising.
a) at least one downloadable printable wrist circumference measurer; and
b) at least one downloadable printable watch lug width measurer.
